(19) United States
(12) Patent Application Publication
(10) Pub. No.: US 2022/0079696 A1
(43) Pub. Date: Mar. 17, 2022

(54) SYSTEMS FOR COUPLING AND STORING AN IMAGING INSTRUMENT

(71) Applicant: Intuitive Surgical Operations, Inc., Sunnyvale, CA (US)

(72) Inventors: Sarah A. Nichols, Santa Clara, CA (US); Lucas S. Gordon, Mountain View, CA (US); Andrew J. Hazelton, San Carlos, CA (US)

(21) Appl. No.: 17/471,418

(22) Filed: Sep. 10, 2021

Related U.S. Application Data
(60) Provisional application No. 63/077,059, filed on Sep. 11, 2020.

(51) Int. Cl. A61B 34/00; A61B 34/20; A61B 34/37

(52) U.S. Cl. CPC A61B 34/70; A61B 2034/301; A61B 34/37; A61B 34/20

(57) ABSTRACT

An imaging coupler comprises an elongate device connector configured to couple to an elongate device. The imaging coupler further comprises an instrument connector configured to couple to an imaging instrument. The imaging instrument is configured to be slidably received within a lumen of the elongate device. The imaging coupler further comprises a body portion extending between the elongate device connector and the instrument connector. The imaging coupler further comprises a tubular member coupled to the instrument connector and extending within the body portion. The instrument connector is movable in parallel with a longitudinal axis of the tubular member while the elongate device is coupled to the elongate device connector.

### CROSS-REFERENCE TO RELATED APPLICATIONS

This application claims priority to and the benefit of U.S. Provisional Application 63/077,059 filed Sep. 11, 2020, which is incorporated by reference herein in its entirety.

### FIELD

Examples described herein relate to systems for coupling and storing an imaging instrument, such as systems for coupling the imaging instrument to an elongate catheter via an imaging coupler and for temporarily storing the imaging instrument during a medical procedure.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert minimally invasive medical tools to reach a target tissue location. Minimally invasive medical tools include instruments such as therapeutic, diagnostic, biopsy, and surgical instruments. Minimally invasive medical tools may also include imaging instruments such as endoscopic instruments. Imaging instruments provide a user with a field of view within the patient anatomy. Some minimally invasive medical tools and imaging instruments may be teleoperated or otherwise computer-assisted.

### SUMMARY

Various features may improve the effectiveness of minimally invasive imaging instruments including coupling members that allow controlled movement and temporary storage systems for use during a medical procedure. The following presents a simplified summary of various examples described herein and is not intended to identify key or critical elements or to delineate the scope of the claims.

Consistent with some examples, an imaging coupler is provided. The imaging coupler includes a catheter connector configured to couple to a catheter. The imaging coupler further includes an instrument connector configured to couple to an imaging instrument. The imaging instrument is configured to be slidably received within a lumen of the catheter. The imaging coupler further includes a body portion extending between the catheter connector and the instrument connector. The imaging coupler further includes a tubular member coupled to the instrument connector and extending within the body portion. The instrument connector is movable in parallel with a longitudinal axis of the tubular member while the catheter is coupled to the catheter connector.

Consistent with some examples, a system is provided. The system includes an imaging instrument configured to be slidably received within a lumen of a catheter. The system further includes an imaging coupler including proximal and distal portions. The imaging coupler includes a catheter connector configured to couple to the catheter. The imaging coupler further includes an instrument connector configured to couple to the imaging instrument. The imaging coupler further includes a body portion extending between the catheter connector and the instrument connector. The imaging coupler further includes a tubular member coupled to the instrument connector and extending within the body portion. The instrument connector is movable in parallel with a longitudinal axis of the tubular member while the catheter is coupled to the catheter connector.

Consistent with some examples, an imaging coupler is provided. The imaging coupler includes a catheter connector configured to couple to a catheter. The imaging coupler further includes an instrument connector configured to couple to an imaging instrument. The imaging instrument is configured to be slidably received within a lumen of the catheter. The imaging coupler further includes a body portion coupled to the catheter connector. The imaging coupler further includes a housing coupled to the instrument connector. The housing includes an inner surface defining a cavity configured to slidably receive the body portion. The instrument connector is movable in parallel with a longitudinal axis of the body portion while the catheter is coupled to the catheter connector.

Consistent with some examples, a system is provided. The system includes an imaging instrument configured to be slidably received within a lumen of a catheter. The system further includes an imaging coupler including proximal and distal portions. The imaging coupler includes a catheter connector configured to couple to the catheter. The imaging coupler further includes an instrument connector configured to couple to the imaging instrument. The imaging coupler further includes a body portion coupled to the catheter connector. The imaging coupler further includes a housing coupled to the instrument connector. The housing includes an inner surface defining a cavity configured to slidably receive the body portion. The instrument connector is movable in parallel with a longitudinal axis of the body portion while the catheter is coupled to the catheter connector.

Consistent with some examples, a storage device is provided. The storage device is configured to be coupled to a robotic-assisted manipulator and is configured to receive an imaging instrument. The storage device includes a proximal portion including a rim defining an opening. The opening includes a first perimeter. The storage device further includes an elongate portion extending distally from the proximal portion. The elongate portion includes a second perimeter, and the first perimeter is greater than the second perimeter. When the imaging instrument is received by the storage device, the imaging instrument is in an unbent configuration.

Consistent with some examples, a medical system is provided. The medical system includes a robotic-assisted manipulator and an imaging instrument including a proximal end and a distal end. The proximal end is configured to be coupled to the robotic-assisted manipulator, and the distal end is configured to be removably received within a catheter. The medical system further includes a storage device coupled to the robotic-assisted manipulator. The storage device is configured to receive the imaging instrument. The storage device includes a proximal portion including a rim defining an opening. The opening includes a first perimeter. The storage device further includes an elongate portion extending distally from the proximal portion. The elongate portion includes a second perimeter, and the first perimeter is greater than the second perimeter. When the imaging instrument is received by the storage device, the imaging instrument is in an unbent configuration.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 illustrates a robotic-assisted medical system according to some examples.

FIG. 2A illustrates an imaging system according to some examples.

FIG. 2B illustrates an imaging coupler between an imaging system and a catheter system according to some examples.

FIG. 3A illustrates an imaging system according to some examples.

FIG. 3B illustrates an imaging coupler of the imaging system of FIG. 3A according to some examples.

FIGS. 4A and 4B illustrate an imaging coupler with a biasing member coupled to a tubular member according to some examples.

FIGS. 5A and 5B illustrate an imaging coupler with a tubular member including a notched distal ring according to some examples.

FIGS. 5C-5E illustrate a projection in a lumen of the body portion of FIG. 5A and a notched ring of the tubular member of FIG. 5A according to some examples.

FIGS. 6A and 6B illustrate an imaging coupler with a threaded member releasably engageable with a tubular member according to some examples.

FIGS. 7A and 7B illustrate an imaging coupler with a threaded member releasably engageable with a tubular member coupled to a biasing member according to some examples.

FIGS. 8A and 8B illustrate an imaging coupler with a plunger according to some examples.

FIGS. 9A and 9B illustrate an imaging coupler with a threaded member releasably engageable with a body portion according to some examples.

FIGS. 10A and 10B illustrate an imaging coupler with a threaded housing and body portion according to some examples.

FIGS. 11A and 11B illustrate an imaging coupler with a body portion with ratchet teeth and a housing with a ratchet key according to some examples.

FIGS. 12A and 12B illustrate an imaging coupler with a body portion with a magnet and a housing with a magnet according to some examples.

FIGS. 13A and 13B illustrate an imaging coupler with a tubular member including a distal tab according to some examples.

FIG. 13C illustrates a projection in a lumen of the body portion of FIG. 13A and a tab of the tubular member of FIG. 13A according to some examples.

FIGS. 14A and 14B illustrate an imaging coupler with an instrument connector that is threadably engageable with a body portion of the imaging coupler according to some examples.

FIGS. 15A and 15B illustrate an imaging coupler with a key releasably engageable with a tubular member of the imaging coupler according to some examples.

FIGS. 16A-16C illustrate an imaging coupler with an instrument connector releasably engageable with a body portion of the imaging coupler by a locking member according to some examples.

FIG. 17A illustrates a storage device coupled to the robotic-assisted medical system of FIG. 1 according to some examples.

FIG. 17B illustrates an instrument positioned within the storage device of FIG. 17A according to some examples.

FIG. 18A illustrates a side view of a proximal portion of the storage device of FIG. 17A according to some examples.

FIG. 18B illustrates a rim and an opening of the storage device of FIG. 17A according to some examples.

FIG. 18C illustrates an attachment member of the storage device of FIG. 17A according to some examples.

FIG. 19 is a simplified diagram of a robotic-assisted medical system according to some examples.

FIG. 20A is a simplified diagram of a medical instrument system according to some examples.

FIG. 20B is a simplified diagram of a medical instrument with an extended medical tool according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

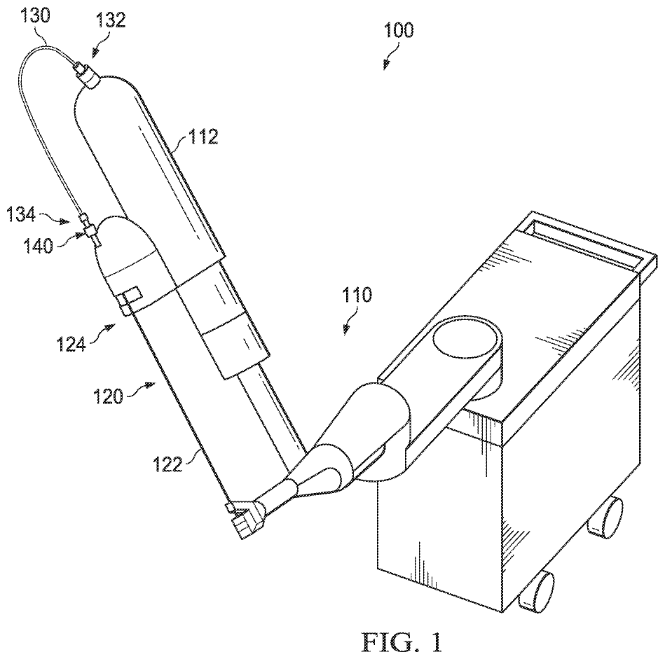

FIG. 1 illustrates a robotic-assisted medical system 100 according to some examples. The robotic-assisted medical system 100 may be suitable for use in surgical, diagnostic, therapeutic, and/or biopsy procedures. As shown in FIG. 1, the medical system 100 may include a manipulator assembly 110 for operating a medical instrument 120 in performing various procedures on a patient. The medical instrument 120 may include an elongate device 122, such as a flexible catheter. The medical instrument 120 may receive an imaging instrument 130, such as an endoscopic imaging instrument. The imaging instrument 130 may be known as a vision probe. A proximal portion 132 of the imaging instrument 130 may be coupled to a manipulator arm 112 of the manipulator assembly 110. A portion 134 of the imaging instrument 130 may be removably coupled to an imaging coupler 140, which may be known as a vision probe adapter. A proximal portion 124 of the medical instrument 120 may also be removably coupled to the imaging coupler 140. For example, the elongate device 122 may be removably coupled to the imaging coupler 140.

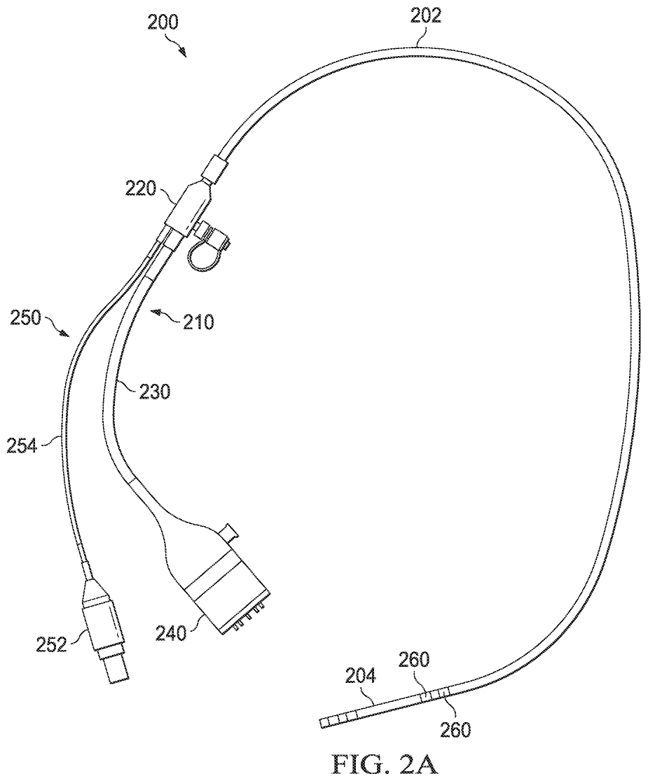

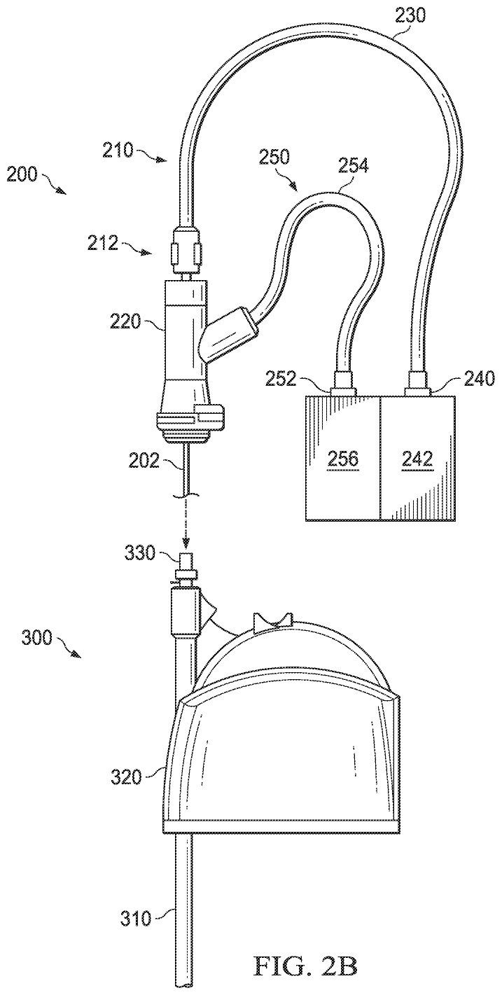

FIG. 2A illustrates an imaging system 200. The imaging system 200 may include an imaging instrument 210 (e.g., the imaging instrument 130) that may be delivered into an anatomy through a catheter (e.g., the medical instrument 120 and/or the elongate device 122). The imaging instrument 210 may include an elongate flexible shaft 202 coupled at a distal end to a rigid or semi-rigid tubular portion 204. In some examples, the elongate flexible shaft 202 may include a long, hollow tube reinforced with steel wire braiding and enclosed within plastic which can be treated by a reflow process. A proximal end of the elongate flexible shaft 202 may be coupled to an imaging coupler 220 (e.g., the imaging coupler 140). The imaging instrument 210 may further include an imaging cable 230. The imaging cable 230 may be coupled to the imaging coupler 220 and may be coupled at a proximal end to an imaging system adapter 240. The imaging system adapter 240 may be coupled to an image processing system (e.g., an image processing system 242 shown in FIG. 2B).

The imaging system 200 may also include a fluid supply system 250. The fluid supply system 250 may include a fluid system adapter 252, which may be coupled to the imaging coupler 220 by tubing 254. The fluid system adapter 252 may be coupled to a fluid delivery system (e.g., a fluid delivery system 256 shown in FIG. 2B). The fluid supply system 250 may be used to clean a lens of a camera, which may be part of and/or inserted into the imaging instrument 210. In one example, the fluid supply system 250 includes a system of pumps and valves, providing for automated, semi-automated, or user-actuated camera cleaning. In an alternative example, the fluid supply system 250 includes a manually operated fluid delivery device (e.g., a syringe). The fluid may be inserted through the fluid system adapter 252 and the tubing 254 for camera cleaning. The fluid system adapter 252, the tubing 254, and/or the imaging coupler 220 may include a set of seals or a luer-activated valve to provide for distal fluid flow while preventing leakage of fluid from the fluid system adapter 252. The fluid may be a liquid, such as saline, and/or a gas. Various camera cleaning systems are disclosed, for example, in International Application No. WO 2016/025465, filed on Aug. 11, 2015, entitled “Systems and Methods for Cleaning an Endoscopic Instrument,” International Application No. WO 2016/040128, filed on Sep. 3, 2015, entitled “Devices, Systems, and Methods Using Mating Catheter Tips and Tools,” and International Application No. WO2019/099396, filed on Nov. 13, 2018, entitled “Systems and Methods for Cleaning Endoscopic Instruments,” each of which is incorporated by reference herein in its entirety.

In some examples, the imaging system 200 includes a keying structure 260, which may be coupled to the elongate flexible shaft 202. In one example, the keying structure 260 may be disposed along a distal portion of the shaft 202, for example, at a location that is proximal of a distal steerable portion of the shaft 202. The keying structure 260 may couple with and/or be received within a groove structure (not shown) in a lumen of a catheter within which the shaft 202 may be received. The keying structure 260 may prevent the shaft 202 from rotating about its longitudinal axis independently from the catheter while the shaft 202 is within the lumen of the catheter.

FIG. 2B illustrates the imaging coupler 220 positioned between the imaging system 200 and a catheter system 300. In some examples, the imaging coupler 220 may be coupled to the imaging instrument 210 and to a catheter 310 of the catheter system 300. The catheter system 300 may include the catheter 310 (e.g., the medical instrument 120 and/or the elongate device 122), a catheter housing 320, and a catheter port 330. The catheter 310 may be coupled to and extend from the catheter housing 320. In some examples, the catheter housing 320 may include the catheter port 330, and the catheter 310 may be coupled to the imaging coupler 220 via the catheter port 330. Additionally or alternatively, the catheter 310 may couple to the imaging coupler 220 directly. For example, a proximal end of the catheter 310 may couple to the imaging coupler 220. In some examples, the imaging instrument 210 may extend through the imaging coupler 220 and through the catheter 310.

For example, the shaft 202 of the imaging instrument 210 may extend through the imaging coupler 220 until a coupling portion 212 of the imaging instrument 210 is coupled to the imaging coupler 220. When the catheter 310 is coupled to the imaging coupler 220, the shaft 202 may extend through a lumen of the catheter 310 while the catheter 310 is coupled to the imaging coupler 220. When the catheter 310 is decoupled from the imaging coupler 220, the shaft 202 may still extend within the lumen of the catheter 310. In examples when the imaging coupler 220 is coupled to the catheter port 330, the shaft 202 of the imaging instrument 210 may extend through the imaging coupler 220, through a lumen of the catheter port 330, and through the lumen of the catheter 310. The imaging instrument 210 may be communicatively coupled to processors of the image processing system 242 via the cable 230. The cable 230 may convey power, image data, instruction signals, or the like, from the imaging instrument 210 to the image processing system 242 and/or from the image processing system 242 to the imaging instrument 210.

In some examples, the imaging instrument 210 may be a bronchoscope, which may be coupled to the imaging coupler 220. The bronchoscope may include a sheath through which a camera of the bronchoscope may extend. In some examples, the bronchoscope sheath may be the catheter 310.

The fluid supply system 250 may be coupled to the catheter 310 via the imaging coupler 220. In examples when the imaging instrument 210 and the catheter 310 are coupled to the imaging coupler 220, the fluid supply system 250 may deliver fluid to a distal end of the catheter 310 to, for example, clean a lens of a camera of the imaging instrument 210. The fluid may be delivered from the fluid delivery system 256, through the tubing 254, through the imaging coupler 220, through a fluid channel of the catheter 310, and to the distal end of the imaging instrument 210. In some examples, the fluid may be delivered through a channel (not shown) positioned between the catheter 310 and the imaging instrument 210.

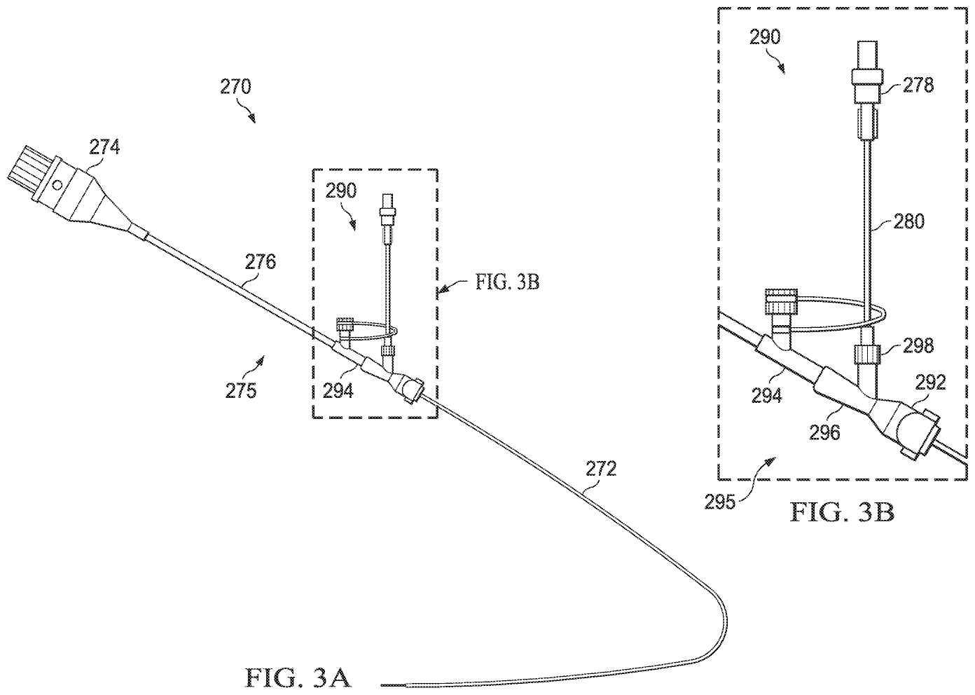

FIGS. 3A and 3B illustrate an alternative imaging system 270, which may be substantially similar to the imaging system 200, except where described below. In a similar manner to the imaging system 200, the imaging system 270 may include an imaging instrument 275. The imaging instrument 275 may include an elongate flexible shaft 272, which may be coupled to an imaging system adapter 274 via a cable 276 and may be coupled to a fluid system adapter 278 via tubing 280. In the example of FIG. 2A, it may be difficult to clean or sterilize components of the imaging system 200, such as the imaging coupler 220, the tubing 254, and/or the fluid system adapter 252. Thus, the imaging coupler 220 may be removeable for cleaning, sterilization, and/or disposal and replacement. Alternatively, as illustrated in FIGS. 3A and 3B, the imaging system 270 may include an imaging coupler 290. The imaging coupler 290 may include a cable adapter 294 which may be coupled to the cable 276. The imaging coupler 290 may also include an imaging probe adapter 295, which may include a body 296, a connector 292, a tubing connector 298, the tubing 280, and the fluid system adapter 278, as described below.

A distal end of the imaging coupler 290 may include the connector 292, which may allow for fast and easy removeable coupling of the cable adapter 294 to a medical device, such as, for example, the medical instrument 120. The imaging coupler 290 may be connected to the tubing 280 in a Y-type fashion. The fluid system adapter 278, the tubing 280, and/or the imaging coupler 290 may include one or more seals and/or a luer-activated valve to provide for fluid flow distally and to prevent leakage of fluid from the fluid system adapter 278. A body 296 of the imaging coupler 290 may be detachable from the cable adapter 294 using a threaded attachment, a removable press fit, a magnetic coupling, and/or the like. In some examples, the cable adapter 294 and/or the imaging coupler 290 may be removable from the imaging instrument 275. This may allow for the cable adapter 294 and/or the imaging coupler 290 to be separately removable for cleaning, sterilization, and/or disposal and replacement with a clean and/or sterile component. In some examples, the cable adapter 294 and/or the imaging coupler 290 may be single use components. Additionally or alternatively, the tubing 280 and/or the fluid system adapter 278 may be removable from the body 296 at the tubing connector 298. This may allow for the tubing 280 and/or the fluid system adapter 278 to be removable for cleaning, sterilization, disposal and/or replacement.

Various additional details regarding imaging systems are disclosed, for example, in International Application No. WO 2019/125581, filed on Oct. 5, 2018, entitled “Imaging Systems and Methods of Use” and International Application No. WO 2019/099396, filed on Nov. 13, 2018, entitled “Systems and Methods for Cleaning Endoscopic Instruments,” each of which is incorporated by reference herein in its entirety.

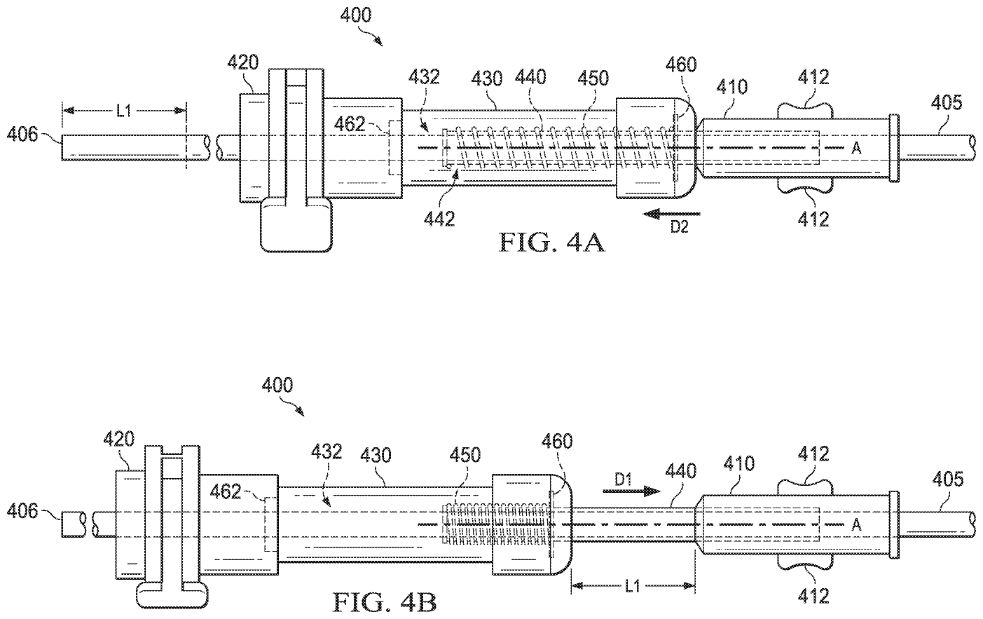

The following discussion will be made with reference to illustrative imaging couplers. Various examples of imaging couplers are provided in FIGS. 4A-12B. The imaging couplers discussed below allow for an insertion distance of an imaging instrument (e.g., the imaging instrument 210) to be adjusted while maintaining a position of a catheter (e.g., the catheter 310, which may be an elongate device) within which the imaging instrument is inserted. The insertion distance may also be adjusted while maintaining a connection between the imaging coupler and the catheter. Adjusting the insertion distance may allow for an image captured by the imaging instrument to be refocused, re-saturated, or otherwise adjusted. For example, the imaging instrument may be retracted to increase the distance between the imaging instrument and a target location until the captured image comes into focus. This may allow the user to confirm whether the imaging instrument is pointed at the target location without moving the catheter and without disconnecting the catheter from the imaging coupler. Additionally, because the catheter remains connected to the imaging coupler, when fluid is introduced through the imaging coupler and through the catheter to clean a lens of the imaging instrument, for example, the fluid does not leak out of the imaging coupler. In examples when the imaging instrument 210 is a bronchoscope, the imaging couplers discussed below may allow for an insertion distance of a camera of the bronchoscope to be adjusted while maintaining a position of a sheath of the bronchoscope within which the camera is inserted.

In some examples, each of the following imaging couplers may couple to the imaging instrument via an instrument connector and may couple to the catheter via a catheter connector, which may be an elongate device connector. The imaging couplers may also include a body portion, which may extend between the instrument connector and the catheter connector. The instrument connector may move in a proximal direction and a distal direction relative to the body portion, which may cause the imaging couplers to move between a collapsed configuration and an extended configuration. Each of the following imaging couplers may be used as the imaging coupler 140 and/or the imaging coupler 220.

FIG. 4A provides a side view of an imaging coupler 400 in a collapsed configuration, and FIG. 4B provides a side view of the imaging coupler 400 in an extended configuration. The imaging coupler 400 may couple to an imaging instrument 405 (e.g., the imaging instrument 130, 210) via an instrument connector 410. The connection between the imaging instrument 405 and the instrument connector 410 may be a threaded connection, an adhesive connection, a welded connection, or any other suitable connection. Additionally or alternatively, the instrument connector 410 may be coupled to a variety of one or more other instruments including, for example, a biopsy instrument, an ultrasound instrument, or an electromagnetic instrument. The imaging coupler 400 may couple to the catheter 310 via a catheter connector 420. The connection between the catheter 310 and the catheter connector 420 may be a threaded connection, an adhesive connection, a welded connection, or any other suitable connection. The imaging instrument 405 may extend through the instrument connector 410, through a tubular member 440, through the catheter connector 420, and within the catheter 310.

The imaging coupler 400 may allow for an insertion distance of the imaging instrument 405 to be adjusted without moving the catheter 310. An adjustment in insertion distance may correspond to the longitudinal movement of a distal end 406 of the imaging instrument 405 relative to the catheter 310.

The instrument connector 410 of the imaging coupler 400 may be moved in a proximal direction D1, which causes the distal end 406 of the imaging instrument 405 to retract within the lumen of the catheter 310. For example, a user may grip one or more tabs 412 and pull the instrument connector 410 in the proximal direction D1. In some examples, the user may pull the instrument connector 410 a distance L1 in the proximal direction D1. When the instrument connector 410 is pulled the distance L1, the distal end 406 of the imaging instrument 405 is moved the distance L1 in the proximal direction D1. In other examples, when the instrument connector 410 is pulled the distance L1, the distal end 406 of the imaging instrument 405 may be moved a distance less than the distance L1 in the proximal direction D1. In other examples, when the instrument connector 410 is pulled the distance L1, the distal end 406 of the imaging instrument 405 may be moved a distance greater than the distance L1 in the proximal direction D1. In some examples, the distal end 406 of the imaging instrument 405 is retracted from a position distal of a distal end of the catheter 310 to a position within the lumen of the catheter 310. In other examples, the distal end 406 of the imaging instrument 405 remains within the lumen of the catheter 310 and is retracted from a position proximal to the distal end of the catheter 310 to a more proximal position within the lumen of the catheter 310. In some examples, the instrument connector 410 is retracted while the catheter 310 remains connected to the imaging coupler 400. For example, the catheter 310 remains connected to a catheter connector 420 of the imaging coupler 400 as the instrument connector 410 is moved.

As discussed above, adjusting the insertion distance of the imaging instrument 405 may allow for an image captured by the imaging instrument 405 to be refocused, re-saturated, or otherwise adjusted. For example, when the catheter 310 is positioned at a target location and the imaging instrument 405 is at a most distal position, an image of the target location captured by the imaging instrument 405 may be fuzzy and out of focus. The image may be refocused by adjusting the distance between the distal end 406 of the imaging instrument 405 and the target location. As discussed above, the imaging instrument 405 may be retracted to increase the distance between the distal end 406 and the target location. The imaging instrument 405 may be retracted until the image comes into focus. This may allow the user to confirm the position of the catheter 310 with respect to the target location, and the user may make additional positional adjustments of the catheter 310 as needed.

In some examples, the instrument connector 410 may be moved 1 cm in the proximal direction D1, which may be a maximum retraction distance for the instrument connector 410. In other examples, the maximum retraction distance may be 0.5 cm, 0.25 cm, 1.5 cm, or any other suitable distance. In some examples, the maximum retraction distance may be the distance L1. Additionally, the instrument connector 410 may be retracted and held at any position between a fully inserted position (e.g., when the imaging coupler 400 is in the collapsed configuration) and a fully retracted position (e.g., when the imaging coupler 400 is in the extended configuration).

In some examples, the distal end 406 of the imaging instrument 405 may be retracted within the lumen of the catheter 310 to determine whether the image needs to be refocused or whether a lens of the imaging instrument 405 needs to be cleaned. This determination may be made by the user and/or by the robotic-assisted medical system, such as by a control system, an image processing system, and/or another control and/or processing system. For example, if the image captured by the imaging instrument 405 is out of focus, the distal end 406 of the imaging instrument 405 may be retracted into the lumen of the catheter 310. If the image remains out of focus, then a determination may be made that the lens of the imaging instrument 405 should be cleaned. To clean the lens, fluid may be supplied by the fluid supply system 250. When the imaging instrument 405 is retracted within the catheter 310, the connection maintained between the catheter 310 and the catheter connector 420 may help prevent any fluid leakage.

In some examples, the imaging coupler 400 also includes a body portion 430 and a tubular member 440 that is movable relative to the body portion 430. The body portion 430 may include a cavity 432, and the tubular member 440 extends within the cavity 432 and is movable within the cavity 432 longitudinally along a longitudinal axis A of the tubular member 440. The body portion 430 may extend between the instrument connector 410 and the catheter connector 420. In some examples, the body portion 430 may have a cross-section of any of a variety of shapes including, for example, circular, rectangular, or triangular. The body portion 430 may be coupled to the catheter connector 420 by a threaded connection, an adhesive connection, a welded connection, or any other suitable connection.

In some examples, a distal end 442 of the tubular member 440 may remain within the cavity 432 when the imaging coupler 400 is in and moves between the collapsed configuration and the extended configuration. For example, as shown in FIG. 4B, when the imaging coupler 400 is in the extended configuration, the instrument connector 410 is spaced from the body portion 430. In some examples, the instrument connector 410 may be pulled back from the body portion 430 in the proximal direction D1, which may be parallel with the longitudinal axis A of the tubular member 440. When the instrument connector 410 moves in the proximal direction D1, the distal end of the imaging instrument 405 is retracted within the lumen of the catheter 310.

In some examples, the imaging coupler 400 may also include a biasing member 450, such as a spring. The biasing member 450 may be coupled to the tubular member 440. In some examples, the biasing member 450 surrounds the tubular member 440, as seen in FIGS. 4A and 4B. The biasing member 450 may bias the tubular member 440 in a distal direction D2, which biases the imaging coupler 400 toward the collapsed configuration. When the instrument connector 410 is pulled in the proximal direction D1, the pulling force overcomes the biasing force to enable the instrument connector 410 to move in the proximal direction D1. When the instrument connector 410 is released, the biasing member 450 may snap the tubular member 440 and the instrument connector 410 in the distal direction D2, which brings the imaging coupler 400 back to the collapsed configuration. In some examples, when the imaging coupler 400 is in a fully extended configuration, the biasing member 450 is in a fully compressed state. In some examples, when the imaging coupler 400 is in the collapsed configuration, the biasing member 450 is in a non-compressed state. Alternatively, when the imaging coupler 400 is in the collapsed configuration, the biasing member 450 may still be slightly compressed.

In alternative examples, the biasing member 450 may bias the tubular member 440 in the proximal direction D1, which biases the imaging coupler 400 in the extended configuration. When the instrument connector 410 is pushed in the distal direction D2, the pushing force overcomes the biasing force to enable the instrument connector 410 to move in the distal direction D2. When the instrument connector 410 is released, the biasing member may snap the tubular member 440 and the instrument connector 410 in the proximal direction D1, which brings the imaging coupler 400 back to the extended configuration.

The imaging coupler 400 may also include a proximal seal 460 and/or a distal seal 462. The seals 460, 462 may prevent fluid, such as the cleaning fluid, from leaking out of the imaging coupler 400. For example, the proximal seal 460 may provide a seal between the tubular member 440 and the body portion 430, and the distal seal 462 may provide a seal between the catheter connector 420 and the body portion 430. The proximal seal 460 provides the seal between the tubular member 440 and the body portion 430 during motion of the tubular member 440 relative to the body portion 430. For example, when the tubular member 440 moves in the proximal direction D1 or the distal direction D2, the proximal seal 460 maintains the seal between the tubular member 440 and the body portion 430. In some examples, each of the seals 460, 462 may be any of a variety of seals including, for example, an o-ring. The tubular member 440 may extend through the proximal seal 460.

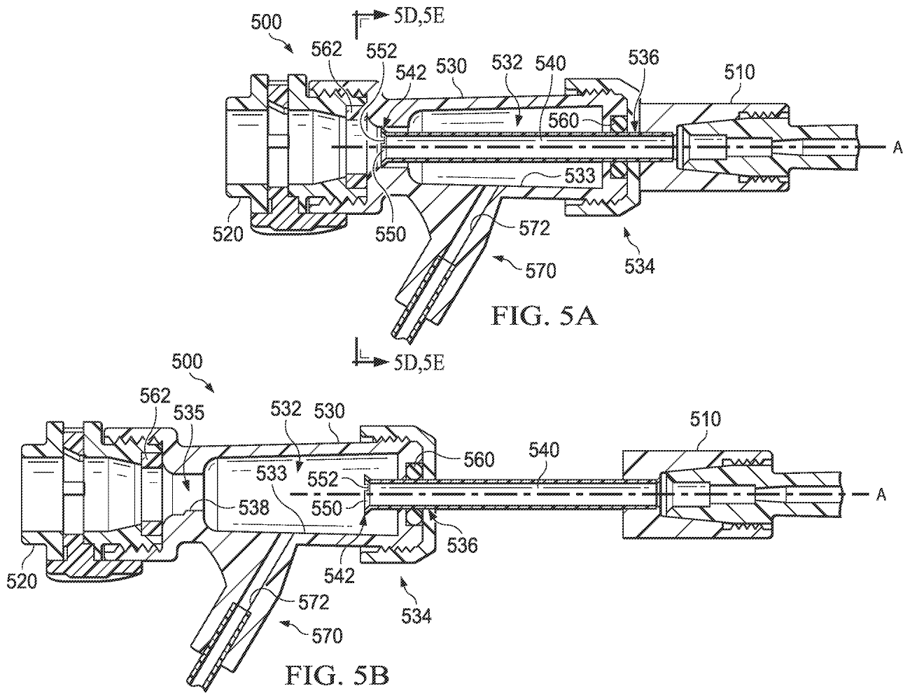

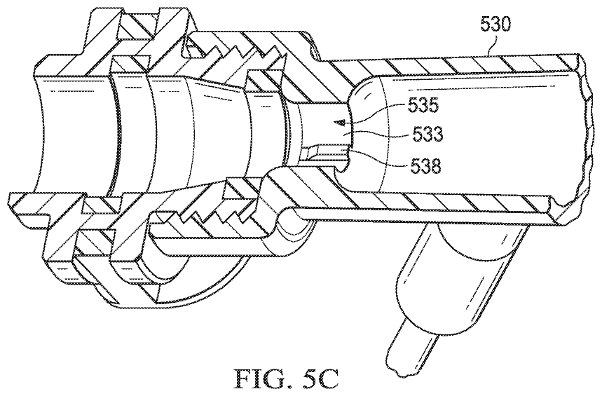

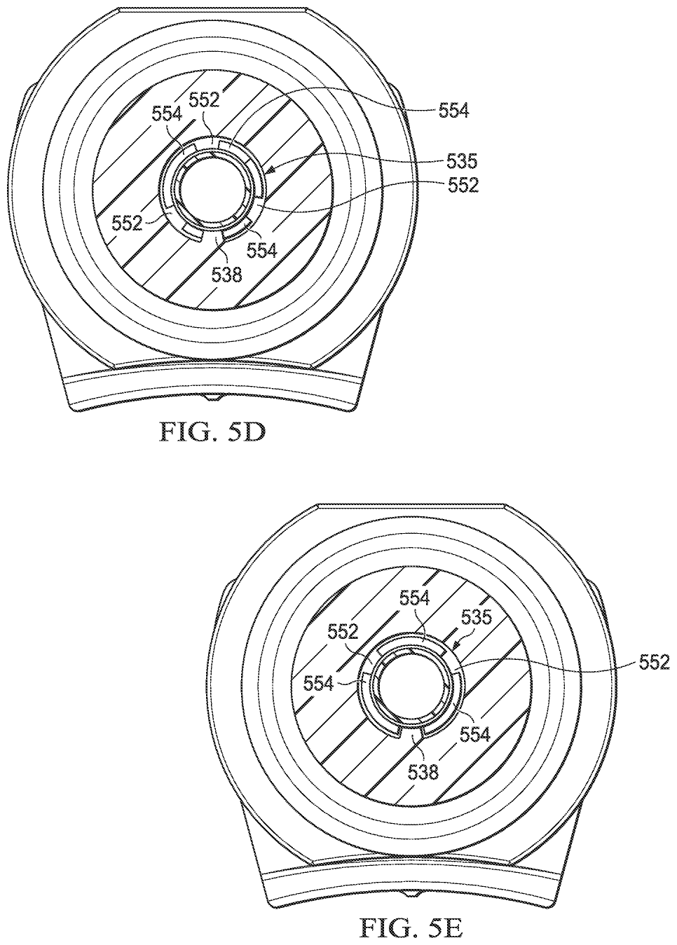

FIG. 5A provides a cross-sectional side view of an imaging coupler 500 in a collapsed configuration, and FIG. 5B provides a cross-sectional side view of the imaging coupler 500 in an extended configuration. As discussed above with respect to FIGS. 4A and 4B, the imaging coupler 500 allows for an insertion distance of the imaging instrument 405 to be adjusted without moving the catheter 310. The imaging coupler 500 includes an instrument connector 510, a catheter connector 520, a body portion 530, a tubular member 540 that is movable relative to the body portion 530, and a ring 550 at a distal end 542 of the tubular member 540. The ring 550 may maintain a position of the tubular member 540 relative to the body portion 530, which will be discussed in further detail below.

The body portion 530 may include a cavity 532, and the tubular member 540 extends within the cavity 532 and is movable within the cavity 532 longitudinally along a longitudinal axis A. The cavity 532 is defined by an inner surface 533 of the body portion 530, and a distal end 542 of the tubular member 540 may remain within the cavity 532 when the imaging coupler 500 is in the extended configuration. The body portion 530 includes a proximal end 534 with a proximal opening 536. The tubular member 540 extends through the proximal opening 536. In some examples, an outer diameter of the ring 550 is greater than an outer diameter of the proximal opening 536. This may help prevent the tubular member 540 from being pulled completely out of the body portion 530. The ring 550 may be flared or tapered.

As discussed above with respect to FIGS. 4A and 4B, the imaging coupler 500 may couple to the imaging instrument 405 via the instrument connector 510, and the imaging coupler 500 may couple to the catheter 310 via the catheter connector 520. As further discussed above, the body portion 530 may extend between and couple to the instrument connector 510 and the catheter connector 520. The imaging instrument 405 may extend along the longitudinal axis A of the tubular member 540 and may extend through the instrument connector 510, through the tubular member 540, through the catheter connector 520, and within the catheter 310 as previously described with respect to FIGS. 4A and 4B. In some examples, the instrument connector 510 may be pulled back from the body portion 530 in a proximal direction that is parallel with a longitudinal axis A of the tubular member 540. When the instrument connector 510 moves in the proximal direction, the distal end of the imaging instrument 405 is retracted within the lumen of the catheter 310. Additionally, the imaging coupler 500 may also include a proximal seal 560 and a distal seal 562, which may be similar to the seals 460 and 462, respectively, as discussed above with respect to FIGS. 4A and 4B.

As seen in FIG. 5B, the body portion 530 includes a distal lumen 535, which is defined by the inner surface 533 of the body portion 530. The distal lumen 535 may be sized to receive the ring 550 of the tubular member 540. In some examples, a projection 538 may extend from the inner surface 533 into the distal lumen 535. The ring 550 may include at least one notch 552. In some examples, the ring 550 includes three notches 552, but any other number of notches may be included (e.g., one notch, two notches, or four notches). The notch 552 may be sized and shaped to fit around the projection 538. The tubular member 540 is rotatable around its longitudinal axis A. In some examples, the tubular member 540 may be rotated until the notch 552 is aligned with the projection 538. When the notch 552 is aligned with the projection 538, the ring 550 may be inserted into/retracted from the distal lumen 535, and the imaging coupler 500 is in an unlocked configuration. When the notch 552 is not aligned with the projection 538, the ring 550 may not be moved within the distal lumen 535, and the imaging coupler 500 is in a locked configuration.

In some examples, when the ring 550 is inserted within the distal lumen 535 and beyond the projection 538, the imaging coupler 500 may be in the collapsed configuration. When the tubular member 540 is rotated to put the imaging coupler 500 in the locked configuration, such as when the notch 552 is not aligned with the projection 538, the projection 538 may maintain the imaging coupler 500 in the collapsed configuration. When the tubular member 540 is rotated to put the imaging coupler 500 in the unlocked configuration, such as when the notch 552 is aligned with the projection 538, the imaging coupler 500 may be moved from the collapsed configuration to the extended configuration.

In some examples, the body portion 530 may include multiple lumens, and each lumen may include a projection. For example, a proximal lumen may be similar to the distal lumen 535 and may be positioned within the cavity 532 proximal to the distal lumen 535. The proximal lumen may include a projection similar to the projection 538. When the body portion 530 includes both the distal lumen 535 and the proximal lumen, the cavity 532 may be divided into three regions—a proximal region, a middle region, and a distal region. The proximal region may be a region proximal of the proximal lumen. The middle region may be a region between the distal lumen 535 and the proximal lumen. The distal region may be a region distal of the distal lumen 535. To move between regions, the notch 552 may need to be aligned with the projection in each lumen (e.g., the projection 538) as discussed above. For example, when the notch 552 is aligned with the projection of the proximal lumen, the imaging coupler 500 is in the unlocked configuration, and the ring 550 of the tubular member 540 may be moved from the proximal region to the middle region and from the middle region to the proximal region. While in the above example, the body portion 530 includes two lumens, each with a projection, the body portion 530 may include any number of lumens with a projection, such as three, four, five, or any other number of lumens. As the number of lumens within the body portion 530 increases, the number of discrete regions within which the tubular member 540 may move increases. For example, when there are three lumens, there may be four discrete regions within which the ring 550 of the tubular member 540 may be located.

FIG. 5C provides a cross-sectional perspective view of the distal lumen 535 when the ring 550 is not within the distal lumen 535. When the ring 550 is inserted within the distal lumen 535 to an axial position that is distal of the projection 538, the tubular member 540, and therefore the ring 550, may be rotated to place the imaging coupler 500 in the locked configuration. For example, FIG. 5D provides a cross-sectional view of the imaging coupler 500 in the locked configuration, and FIG. 5E provides a cross-sectional view of the imaging coupler 500 in the unlocked configuration. In both FIGS. 5D and 5E, the cross-sectional view is provided from a position distal of the distal lumen 535 and looking in the proximal direction.

In some examples, the imaging coupler 500 is in the locked configuration when the ring 550 is inserted within the distal lumen 535 and the notch 552 is not aligned with the projection 538. For example, FIG. 5D shows the ring 550 positioned distally of the projection 538. The tubular member 540 is rotated such that the notch(es) 552 is not aligned with the projection 538. Instead, a fin 554 of the ring 550 is positioned distally in front of the projection 538. When the imaging coupler 500 is in the locked configuration, the tubular member 540 may not be moved in a proximal direction because the fin 554 contacts the projection 538 and prevents the ring 550 from being moved proximally through the distal lumen 535.

In some examples, the imaging coupler 500 is in the unlocked position when the ring 550 is inserted within the distal lumen 535 and the notch 552 is aligned with the projection 538. For example, FIG. 5E shows the ring 550 positioned distally of the projection 538. The tubular member 540 is rotated such that the notch(es) 552 is aligned with the projection 538. When the imaging coupler 500 is in the unlocked configuration, the tubular member 540 may be moved in a proximal direction because the notch 552 fits around the projection 538, and the ring 550 may move proximally through the distal lumen 535.

In some examples, the imaging coupler 500 may also include a biasing member (not shown), such as a spring or any other suitable biasing member similar to the biasing member 450 in FIGS. 4A and 4B. The biasing member may be coupled to the tubular member 540. In some examples, the biasing member may surround the tubular member 540. The biasing member may bias the tubular member 540 in the distal direction, which may bias the imaging coupler 500 toward the collapsed configuration. Alternatively, the biasing member may bias the tubular member 540 in the proximal direction, which may bias the imaging coupler 500 in the extended configuration.

The imaging coupler 500 may also include a fluid connector 570, which may include tubing 572 (e.g., the tubing 254). In some examples, the fluid supply system 250 includes the fluid connector 570. Cleaning fluid may be supplied from the fluid delivery system 256, through the tubing 572 of the fluid connector 570, and into the cavity 532 of the body portion 530. The cleaning fluid may then be supplied to a lens of the imaging instrument 405 and/or to a lens of a camera of the imaging instrument 405 to clean the lens, for example, and remove any visual obstructions on the lens, such as blood, mucus, and/or any other fluids/particles. When the fluid is supplied into the cavity 532, the seals 560, 562 prevent the fluid from leaking out of the imaging coupler 500. If the imaging coupler 500 is in the locked position when fluid is supplied into the cavity 532, the imaging coupler 500 may remain in the locked position, despite the pressure from the fluid. In this locked position, the fin 554 and the projection 538 may prevent the tubular member 540 from being pushed in a proximal direction by a fluid force imparted by the fluid onto the instrument connector 510 to which the tubular member 540 is coupled.

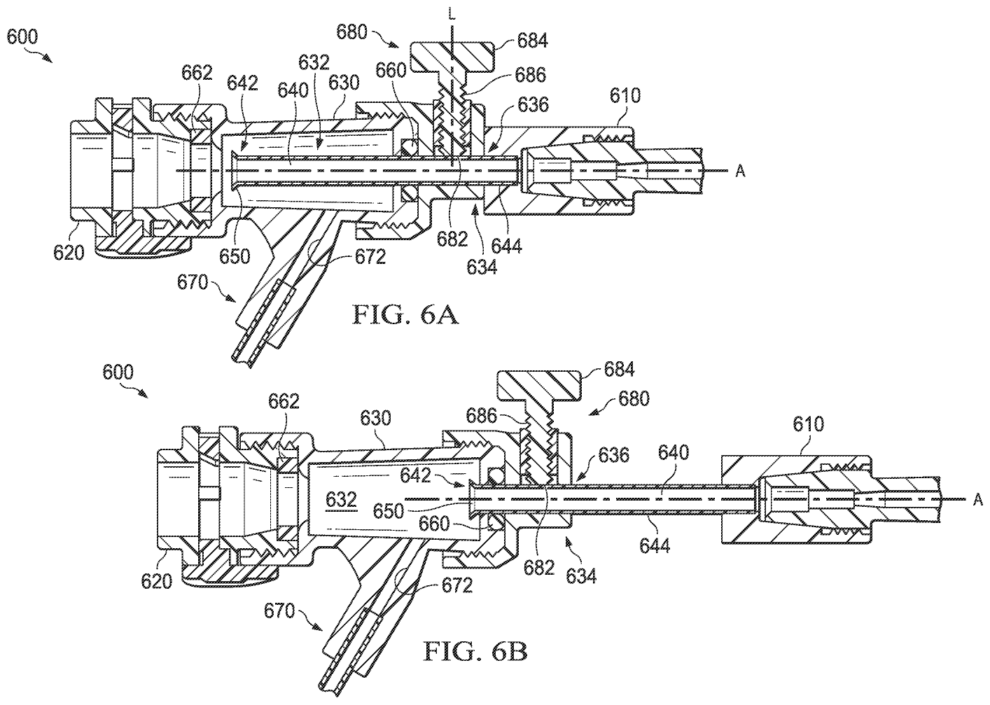

FIG. 6A provides a cross-sectional side view of an imaging coupler 600 in a collapsed configuration, and FIG. 6B provides a cross-sectional side view of the imaging coupler 600 in an extended configuration. As discussed above with respect to FIGS. 4A and 4B, the imaging coupler 600 allows for an insertion distance of the imaging instrument 405 to be adjusted without moving the catheter 310. The imaging coupler 600 includes an instrument connector 610, a catheter connector 620, a body portion 630, a tubular member 640 that is movable relative to the body portion 630, and a threaded member 680. The threaded member 680 may maintain a position of the tubular member 640 relative to the body portion 630, which will be discussed in further detail below.

The body portion 630 may include a cavity 632, and the tubular member 640 extends within the cavity 632 and is movable within the cavity 632 longitudinally along a longitudinal axis A. In some examples, a distal end 642 of the tubular member 640 may remain within the cavity 632 when the imaging coupler 600 is in the extended configuration. The body portion 630 also includes a proximal end 634 with a proximal opening 636. The tubular member 640 extends through the proximal opening 636. In some examples, the tubular member 640 includes a ring 650 at the distal end 642 of the tubular member 640. In some examples, an outer diameter of the ring 650 is greater than an outer diameter of the proximal opening 636 of the body portion 630. This may help prevent the tubular member 640 from being pulled completely out of the body portion 630.

As discussed above with respect to FIGS. 4A and 4B, the imaging coupler 600 may couple to the imaging instrument 405 via the instrument connector 610, and the imaging coupler 600 may couple to the catheter 310 via the catheter connector 620. As further discussed above, the body portion 630 may extend between and couple to the instrument connector 610 and the catheter connector 620. The imaging instrument 405 may extend along the longitudinal axis A of the tubular member 640 and may extend through the instrument connector 610, through the tubular member 640, through the catheter connector 620, and within the catheter 310 as previously described with respect to FIGS. 4A and 4B. In some examples, the instrument connector 610 may be pulled back from the body portion 630 in a proximal direction that is parallel with the longitudinal axis A of the tubular member 640. When the instrument connector 610 moves in the proximal direction, the distal end of the imaging instrument 405 is retracted within the lumen of the catheter 310. Additionally, the imaging coupler 600 may also include a proximal seal 660 and a distal seal 662, which may be similar to the seals 460 and 462, respectively, as discussed above with respect to FIGS. 4A and 4B. The imaging coupler 600 may also include a fluid connector 670, which may be similar to the fluid connector 570 discussed above with respect to FIGS. 5A and 5B.

In some examples, when the imaging coupler 600 is in the collapsed configuration and the threaded member 680 is in contact with the tubular member 640, the threaded member 680 may maintain the imaging coupler 600 in the collapsed position. When the imaging coupler 600 is in the extended configuration and the threaded member 680 is in contact with the tubular member 640, the threaded member 680 may maintain the imaging coupler 600 in the extended position. The threaded member 680 may also be in contact with the tubular member 640 at any other position to maintain the image coupler 600 in an intermediate position between the collapsed configuration and the extended configuration. When the threaded member 680 is not in contact with the tubular member 640, the imaging coupler 600 may be moved between the collapsed configuration and the extended configuration.

In some examples, the threaded member 680 may be a set screw or other screw member. In some examples, the body portion 630 may include the threaded member 680. In other examples, the threaded member 680 may be coupled to the body portion 630. The threaded member 680 is rotatable around its longitudinal axis L. In some examples, a user may rotate the threaded member 680 by rotating a proximal portion 684. The proximal portion 684 may have a larger diameter than a threaded body portion 686 of the threaded member 680. In some examples, a distal end 682 of the threaded member 680 may engage with an outer surface 644 of the tubular member 640. For example, when the threaded member 680 is rotated to a locked position, the distal end 682 may contact the outer surface 644. In some examples, the threaded member 680 is rotated in a clockwise direction to bring the threaded member 680 into the locked position. When the threaded member 680 is in the locked positioned, the imaging coupler 600 is in a locked configuration in which the tubular member 640 is not movable in the proximal or distal directions. The threaded member 680 may then be rotated in a counterclockwise direction to disengage the distal end 682 from the outer surface 644. When the distal end 682 is disengaged from the outer surface 644, the threaded member 680 is in an unlocked position. When the threaded member 680 is in the unlocked position, the imaging coupler 600 is in an unlocked configuration in which the tubular member 640 is movable in the proximal and distal directions.

If the imaging coupler 600 is in the locked position when fluid is supplied into the cavity 632, the imaging coupler 600 may remain in the locked position despite the pressure from the fluid. In this locked position, a friction force between the distal end 682 of the threaded member 680 and the outer surface 644 of the tubular member 640 may prevent the tubular member 640 from being pushed in the proximal direction by a fluid force imparted by the fluid onto the instrument connector 610 to which the tubular member 640 is coupled.

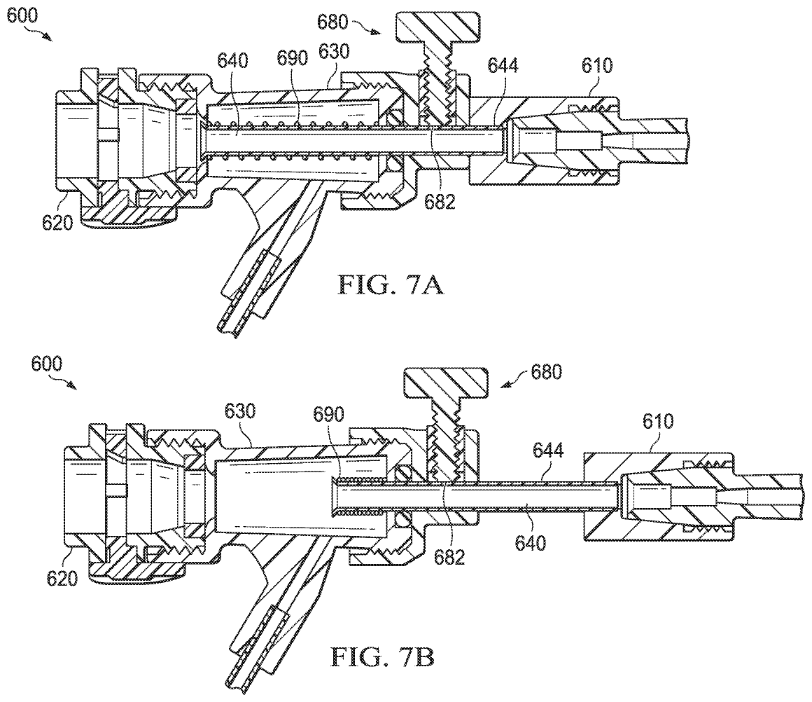

As shown in FIGS. 7A and 7B, in some examples, the imaging coupler 600 may also include a biasing member 690. The biasing member 690 may be a spring or any other suitable biasing member. The biasing member 690 may be coupled to the tubular member 640. In some examples, the biasing member 690 may surround the tubular member 640. The biasing member 690 may bias the tubular member 640 in a distal direction, which may bias the imaging coupler 600 toward the collapsed configuration. When the distal end 682 of the threaded member 680 is disengaged from the outer surface 644 of the tubular member 640 and when the instrument connector 610 is pulled in the proximal direction, the pulling force overcomes the biasing force to enable the instrument connector 610 to move in the proximal direction. When the instrument connector 610 is released, the biasing member 690 may snap the tubular member 640 and the instrument connector 610 in the distal direction, which brings the imaging coupler 600 to the collapsed configuration.

In some examples, the threaded member 680 may be rotated to engage with the tubular member 640 when the imaging coupler 600 is in the extended configuration. For example, when the instrument connector 610 is pulled in the proximal direction, the threaded member 680 may be rotated so that the distal end 682 engages with the outer surface 644 of the tubular member 640. In such examples, the friction force between the distal end 682 and the outer surface 644 overcomes the biasing force and maintains the imaging coupler 600 in the extended configuration. This may prevent the instrument connector 610 from being snapped back in the distal direction by the biasing member 690. When the threaded member 680 is rotated so that the distal end 682 disengages from the outer surface 644, the biasing member 690 may snap the tubular member 640 and the instrument connector 610 in the distal direction, which brings the imaging coupler 600 to the collapsed configuration. In alternative examples, the biasing member 690 may bias the tubular member 640 in a proximal direction, which may bias the imaging coupler 600 toward the extended configuration.

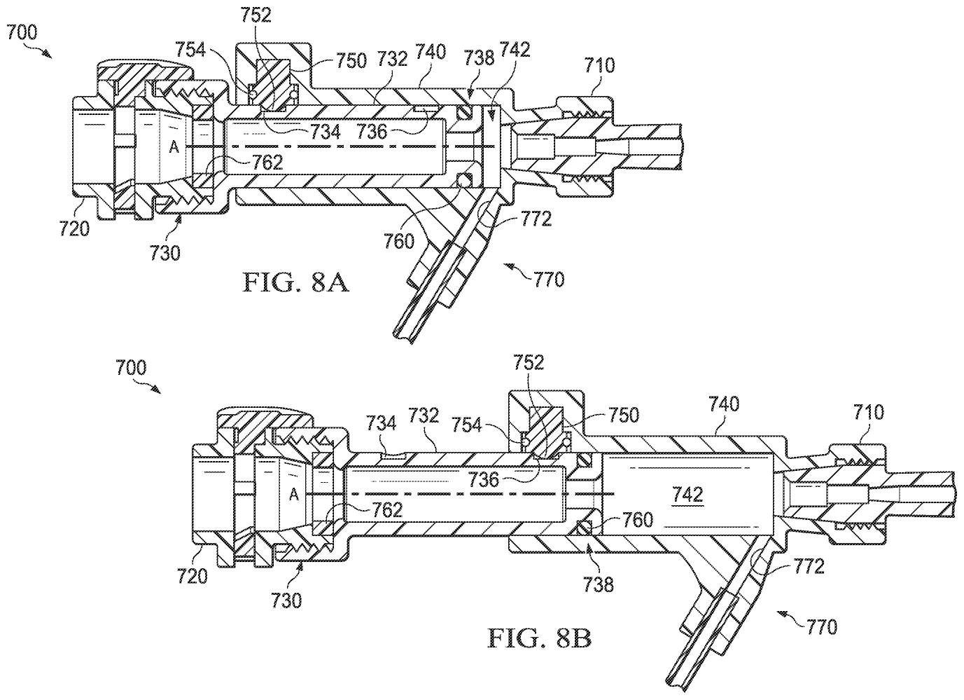

FIG. 8A provides a cross-sectional side view of an imaging coupler 700 in a collapsed configuration, and FIG. 8B provides a cross-sectional side view of the imaging coupler 700 in an extended configuration. As discussed above with respect to FIGS. 4A and 4B, the imaging coupler 700 may allow for an insertion distance of the imaging instrument 405 to be adjusted without moving the catheter 310. The imaging coupler 700 includes an instrument connector 710, a catheter connector 720, a body portion 730, a housing 740 that is movable relative to the body portion 730, and a plunger 750. The plunger 750 may maintain a position of the housing 740 relative to the body portion 730, which will be discussed in further detail below.

The housing 740 may include a cavity 742, and the body portion 730 extends within the cavity 742 and is movable within the cavity 742 longitudinally along a longitudinal axis A. For example, a proximal end 738 of the body portion 730 may remain within the cavity 742 when the imaging coupler 700 is in the extended configuration.

As discussed above with respect to FIGS. 4A and 4B, the imaging coupler 700 may couple to the imaging instrument 405 via the instrument connector 710, and the imaging coupler 700 may couple to the catheter 310 via the catheter connector 720. As further discussed above, the body portion 730 may couple to the catheter connector 720. In some examples, the housing 740 may couple to the instrument connector 710. The imaging instrument 405 may extend along a longitudinal axis A of the body portion 730 and may extend through the instrument connector 710, through the housing 740, through the body portion 730, through the catheter connector 720, and within the catheter 310 as previously described with respect to FIGS. 4A and 4B. The instrument connector 710 may be pulled back from the body portion 730 in a proximal direction that is parallel with the longitudinal axis A of the body portion 730. When the instrument connector 710 moves in the proximal direction, the distal end of the imaging instrument 405 is retracted within the lumen of the catheter 310. Additionally, the imaging coupler 700 may also include a proximal seal 760 and a distal seal 762, which may be similar to the seals 460 and 462, respectively, as discussed above with respect to FIGS. 4A and 4B. The imaging coupler 700 may also include a fluid connector 770, which may be similar to the fluid connector 570 discussed above with respect to FIGS. 5A and 5B.

In some examples, when the plunger 750 is positioned within a distal indentation 734 of the body portion 730, the imaging coupler 700 may be in the collapsed configuration, and the plunger 750 may maintain the imaging coupler 700 in the collapsed configuration. When the plunger 750 is positioned within a proximal indentation 736 of the body portion 730, the imaging coupler 700 may be in the extended configuration, and the plunger 750 may maintain the imaging coupler 700 in the extended configuration. When the plunger 750 is positioned outside of the distal indentation 734 and the proximal indentation 736, the imaging coupler 700 may be moved between the collapsed configuration and the extended configuration.

The plunger 750 may be a ball plunger or any other plunger. The plunger 750 may be coupled to the housing 740. The plunger 750 may include an internal biasing member (not shown) that biases the plunger 750 toward an outer surface 732 of the body portion 730. The plunger 750 may include a distal portion 752 that contacts the outer surface 732. In some examples, the distal portion 752 may have a cross-section of any of a variety of shapes including, for example, circular, rectangular, or triangular. The outer surface 732 may define the distal indentation 734 and/or the proximal indentation 736. The outer surface 732 may define any number of additional indentations, which may be positioned proximal of the distal indentation 734 and/or distal of the proximal indentation 736.

The indentations 734, 736 may be sized and shaped to receive the distal portion 752 of the plunger 750. For example, when the plunger 750 is aligned with the distal indentation 734, the biasing element of the plunger 750 biases the distal portion 752 into the distal indentation 734. When the distal portion 752 is positioned within the distal indentation 734, the imaging coupler 700 may be in the collapsed configuration and in a locked configuration. In some examples, when the instrument connector 710 is moved in a proximal direction, which may be done via an axial pulling force, the axial pulling force overcomes the biasing force imparted by the internal biasing member of the plunger 750. When the axial pulling force overcomes the biasing force, the distal portion 752 is pulled out of the distal indentation 734, and the housing 740 and the plunger 750 move in the proximal direction.

In some examples, the housing 740 may be pulled in the proximal direction until the distal portion 752 of the plunger 750 is biased into the proximal indentation 736. When the distal portion 752 is positioned within the proximal indentation 736, the imaging coupler 700 may be in the extended configuration and in the locked configuration. In some examples, when the instrument connector 710 is moved in a distal direction, which may be done via an axial pushing force, the axial pushing force may overcome the biasing force imparted by the internal biasing member of the plunger 750. When the axial pushing force overcomes the biasing force, the distal portion 752 is pushed out of the proximal indentation 736, and the housing 740 and the plunger 750 move in the distal direction. In some examples, the body portion 730 may include one indentation (e.g., one of the proximal indentation 736 or the distal indentation 734) or may include more than two indentations (e.g., the proximal indentation 736, the distal indentation 734, and one or more other indentations between the proximal indentation 736 and the distal indentation 734).

If the imaging coupler 700 is in the locked position when fluid is supplied into the cavity 742, the imaging coupler 700 may remain in the locked position despite the pressure from the fluid. In this locked position, a friction force between the distal portion 752 of the plunger 750 and the distal indentation 734, for example, may prevent the housing 740 from being pushed in the proximal direction by a fluid force imparted by the fluid onto the instrument connector 710 to which the housing 740 is coupled.

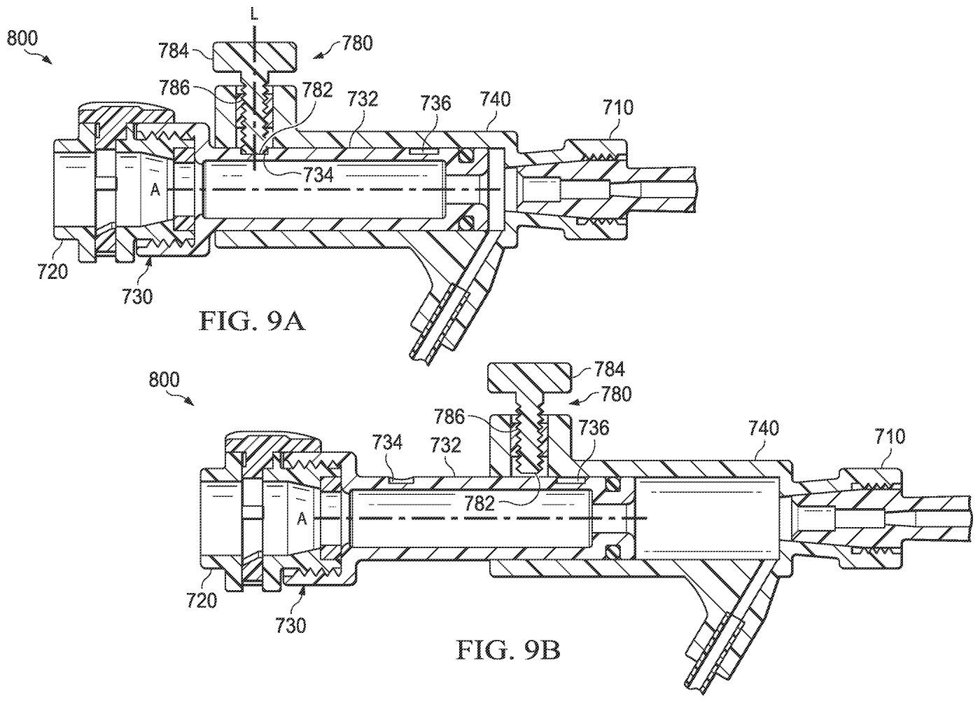

As shown in FIGS. 9A and 9B, in some alternative examples, an imaging coupler 800 may include the instrument connector 710, the catheter connector 720, the body portion 730, the housing 740, and a threaded member 780. The threaded member 780 may be a set screw or other screw member and may be substantially similar to the threaded member 680 described above with respect to FIGS. 6A and 6B. The discussion with respect to the threaded member 680 similarly applies to the threaded member 780, and additional discussion regarding the threaded member 780 will now be made.

The threaded member 780 may maintain a position of the housing 740 relative to the body portion 730. In some examples, when the threaded member 780 is positioned within the distal indentation 734 of the body portion 730, the imaging coupler 700 may be in the collapsed configuration, and the threaded member 780 may maintain the imaging coupler 700 in the collapsed configuration. When the threaded member 780 is positioned within the proximal indentation 736 of the body portion 730, the imaging coupler 700 may be in the extended configuration, and the threaded member 780 may maintain the imaging coupler 700 in the extended configuration. When the threaded member 780 is positioned outside of the distal indentation 734 and the proximal indentation 736 but is in contact with the body portion 730, the position of the housing 740 may be maintained relative to the body portion 730. When the threaded member 780 is positioned outside of the distal indentation 734 and the proximal indentation 736 and is not in contact with the body portion 730, the imaging coupler 700 may be moved between the collapsed configuration and the extended configuration.

The distal end 782 of the threaded member 780 may be sized and shaped to fit within the distal indentation 734 and the proximal indentation 736. When the threaded member 780 is rotated so that the distal end 782 is positioned within the distal indentation 734, the distal end 782 may contact the outer surface 732 of the body portion 730 within the distal indentation 734. When the distal end 782 is positioned within the distal indentation 734, the imaging coupler 800 may be in the collapsed configuration and in the locked configuration. When the threaded member 780 is rotated so that the distal end 782 is positioned outside of the distal indentation 734, the instrument connector 710 may be moved in the proximal direction. The housing 740 may be pulled in the proximal direction until the distal end 782 is aligned with the proximal indentation 736. When the distal end 782 is aligned with the proximal indentation 736, the threaded member 780 may be rotated so that the distal end 782 is positioned within the proximal indentation 736. The distal end 782 may contact the outer surface 732 of the body portion 730 within the proximal indentation 736. When the distal end 782 is positioned within the proximal indentation 736, the imaging coupler 700 may be in the extended configuration and in the locked configuration. In some examples, the threaded member 780 may be rotated while the imaging coupler 700 is in a position between the extended configuration and the collapsed configuration. When the distal end 782 of the treaded member 780 contacts the outer surface 732 of the body portion 730, the imaging coupler 700 may be in an intermediate configuration between the extended configuration and the collapsed configuration. In some examples, the body portion 730 may include one indentation (e.g., one of the proximal indentation 736 or the distal indentation 734) or may include more than two indentations (e.g., the proximal indentation 736, the distal indentation 734, and one or more other indentations between the proximal indentation 736 and the distal indentation 734).

If the imaging coupler 800 is in the locked position when fluid is supplied into the cavity 742, the imaging coupler 800 may remain in the locked position despite the pressure from the fluid. In this locked position, a friction force between the distal end 782 of the threaded member 780 and the distal indentation 734, for example, may prevent the housing 740 from being pushed in the proximal direction by a fluid force imparted by the fluid onto the instrument connector 710 to which the housing 740 is coupled.

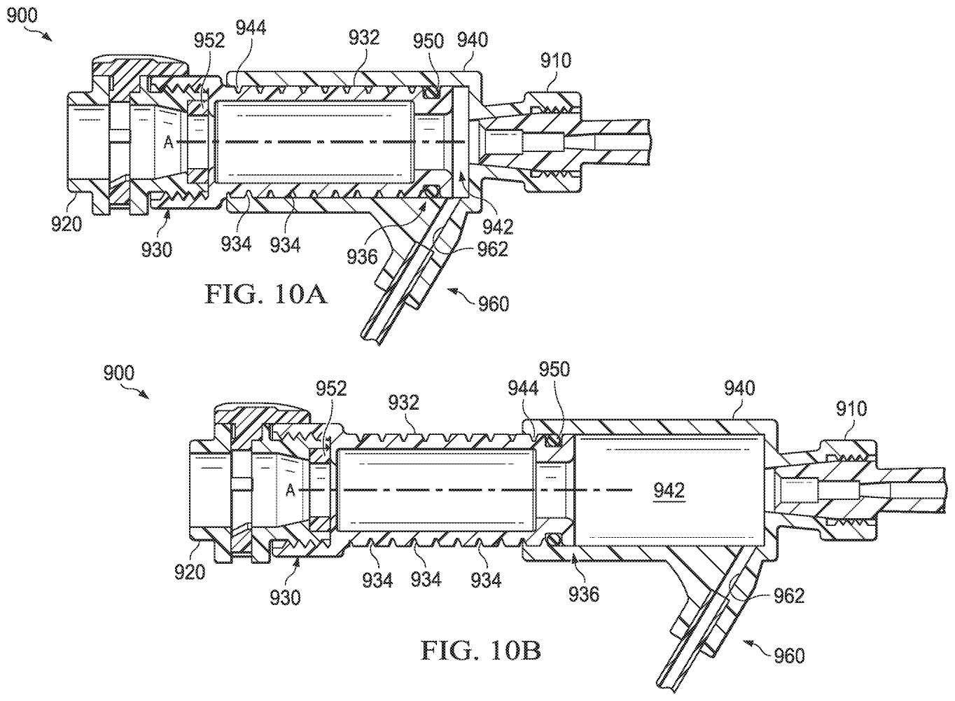

FIG. 10A provides a cross-sectional side view of an imaging coupler 900 in a collapsed configuration, and FIG. 10B provides a cross-sectional side view of the imaging coupler 900 in an extended configuration. As discussed above with respect to FIGS. 4A and 4B, the imaging coupler 900 allows for an insertion distance of the imaging instrument 405 to be adjusted without moving the catheter 310. The imaging coupler 900 includes an instrument connector 910, a catheter connector 920, a body portion 930, and a housing 940 that is movable relative to the body portion 930. In some examples, the body portion 930 may be threadedly engaged with the housing 940. This threaded engagement may maintain a position of the housing 940 relative to the body portion 930, which will be discussed in further detail below.

The housing 940 may include a cavity 942, and the body portion 930 extends within the cavity 942 and is movable within the cavity 942 longitudinally along a longitudinal axis A. For example, a proximal end 936 of the body portion 930 may remain within the cavity 942 when the imaging coupler 900 is in the extended configuration.

As discussed above with respect to FIGS. 4A and 4B, the imaging coupler 900 may couple to the imaging instrument 405 via the instrument connector 910, and the imaging coupler 900 may couple to the catheter 310 via the catheter connector 920. As further discussed above, the body portion 930 may couple to the catheter connector 920. In some examples, the housing 940 may couple to the instrument connector 910. The imaging instrument 405 may extend along a longitudinal axis A of the body portion 930 and may extend through the instrument connector 910, through the housing 940, through the body portion 930, through the catheter connector 920, and within the catheter 310 as previously described with respect to FIGS. 4A and 4B. In some examples, the instrument connector 910 may be pulled back from the body portion 930 in a proximal direction that is parallel with the longitudinal axis A of the body portion 930. When the instrument connector 910 moves in the proximal direction, the distal end of the imaging instrument 405 is retracted within the lumen of the catheter 310. Additionally, the imaging coupler 900 may also include a proximal seal 950 and a distal seal 952, which may be similar to the seals 460 and 462, respectively, as discussed above with respect to FIGS. 4A and 4B. The imaging coupler 900 may also include a fluid connector 960, which may be similar to the fluid connector 570 discussed above with respect to FIGS. 5A and 5B.

In some examples, an outer surface 932 of the body portion 930 may include one or more grooves 934. The grooves may be sized and shaped to receive one or more threads 944, which may be on an inner surface 946 of the housing 940. In alternative examples, the inner surface 946 of the housing 940 may include one or more grooves, and the outer surface 932 of the body portion 930 may include one or more corresponding threads.

In some examples, the housing 940 may be rotated relative to the body portion 930. The housing 940 may be rotated while the body portion 930 remains rotationally stationary. For example, the housing 940 may be rotated in a counterclockwise manner about the axis A of the body portion 930. This counterclockwise rotation moves the housing 940 in the proximal direction, which moves the imaging coupler 900 toward a fully extended configuration. The housing 940 may be rotated in a clockwise manner about the axis A to move the housing 940 in the distal direction. This moves the imaging coupler 900 toward the collapsed configuration.

In some alternative examples, the body portion 930 may be rotated relative to the housing 940. The body portion 930 may be rotated while the housing 940 remains rotationally stationary. For example, the body portion 930 may be rotated in a clockwise manner about the axis A. This clockwise rotation moves the body portion 930 in the distal direction, which moves the imaging coupler 900 toward a fully extended configuration. The body portion 930 may be rotated in a counterclockwise manner about the axis A to move the body portion 930 in the proximal direction. This moves the imaging coupler 900 toward the collapsed configuration.

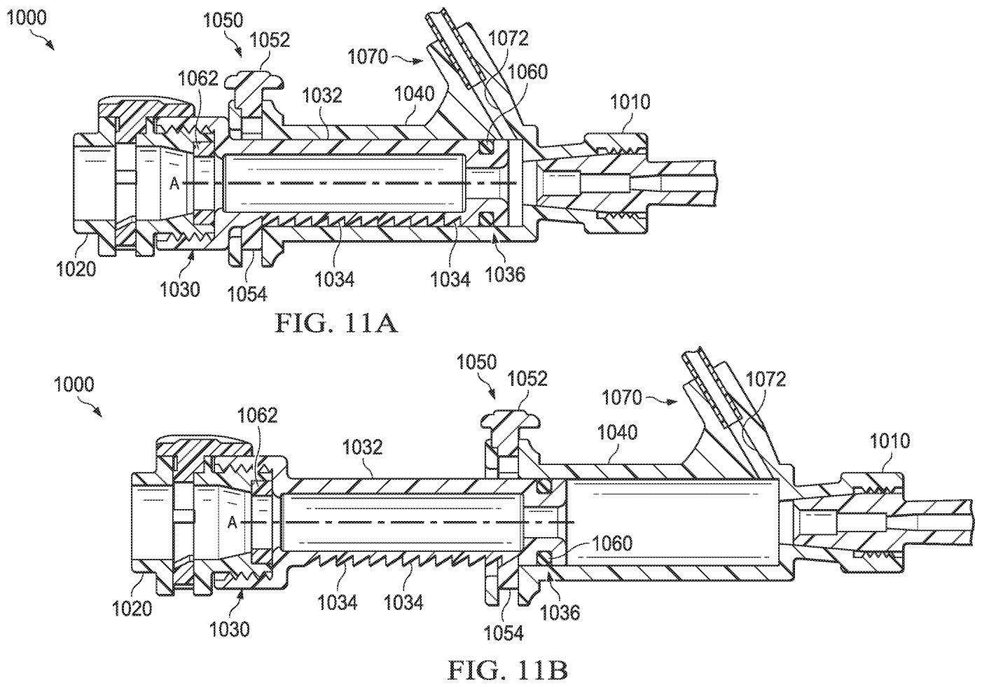

FIG. 11A provides a cross-sectional side view of an imaging coupler 1000 in a collapsed configuration, and FIG. 11B provides a cross-sectional side view of the imaging coupler 1000 in an extended configuration. As discussed above with respect to FIGS. 4A and 4B, the imaging coupler 1000 allows for an insertion distance of the imaging instrument 405 to be adjusted without moving the catheter 310. The imaging coupler 1000 includes an instrument connector 1010, a catheter connector 1020, a body portion 1030, a housing 1040 that is movable relative to the body portion 1030, and a ratchet assembly 1050. The ratchet assembly 1050 may maintain a position of the housing 1040 relative to the body portion 1030, which will be discussed in further detail below.

The housing 1040 may include a cavity 1042, and the body portion 1030 extends within the cavity 1042 and is movable within the cavity 1042 longitudinally along a longitudinal axis A. For example, a proximal end 1036 of the body portion 1030 may remain within the cavity 1042 when the imaging coupler 1000 is in the extended configuration.

As discussed above with respect to FIGS. 4A and 4B, the imaging coupler 1000 may couple to the imaging instrument 405 via the instrument connector 1010, and the imaging coupler 1000 may couple to the catheter 310 via the catheter connector 1020. As further discussed above, the body portion 1030 may couple to the catheter connector 1020. In some examples, the housing 1040 may couple to the instrument connector 1010. The imaging instrument 405 may extend along a longitudinal axis A of the body portion 1030 and may extend through the instrument connector 1010, through the housing 1040, through the body portion 1030, through the catheter connector 1020, and within the catheter 310 as previously described with respect to FIGS. 4A and 4B. In some examples, the instrument connector 1010 may be pulled back from the body portion 1030 in a proximal direction that is parallel with the longitudinal axis A of the body portion 1030. When the instrument connector 1010 moves in the proximal direction, the distal end of the imaging instrument 405 is retracted within the lumen of the catheter 310. Additionally, the imaging coupler 1000 may also include a proximal seal 1060 and a distal seal 1062, which may be similar to the seals 460 and 462, respectively, as discussed above with respect to FIGS. 4A and 4B. The imaging coupler 1000 may also include a fluid connector 1070, which may be similar to the fluid connector 570 discussed above with respect to FIGS. 5A and 5B.

The ratchet assembly 1050 may be coupled to the housing 1040. In other examples, the ratchet assembly 1050 may be integrally formed with the housing 1040. The tab 1052 may be depressed in a direction that is radially inward toward the axis A of the body portion 1030. When the tab 1052 is depressed, the key 1054 may be moved radially outward. In some examples, the tab 1052 may include an internal biasing member (not shown) that biases the tab 1052 in a radially outward direction. For example, when the tab 1052 is not depressed, the tab may remain in an extended position due to a biasing force imparted by the internal biasing member.

In some examples, the ratchet assembly 1050 may be movable between an engaged configuration and a disengaged configuration. In the engaged configuration, the tab 1052 is not depressed, and the key 1054 is engaged with at least one tooth 1034 on the outer surface 1032 of the body portion 1030. When the ratchet assembly 1050 is in the engaged configuration, the housing 1040 is not movable in the proximal direction. Thus, when the ratchet assembly 1050 is in the engaged configuration, the imaging coupler 1000 is in a locked configuration. When the ratchet assembly 1050 is in the disengaged configuration, the tab 1052 is depressed, and the key 1054 is disengaged from the teeth 1034. When the ratchet assembly 1050 is in the disengaged configuration, the housing 1040 is movable in the proximal direction. Thus, when the ratchet assembly 1050 is in the disengaged configuration, the imaging coupler 1000 is in an unlocked configuration. In some examples, when the housing 1040 is pushed in the distal direction, the key 1054 slides over the teeth 1034 even if the tab 1052 is not depressed. Therefore, to move the housing 1040 in the proximal direction, the tab 1052 is depressed, and the housing 1040 is pulled in the proximal direction. To move the housing 1040 in the distal direction, though, the tab 1052 does not need to be depressed (but may still be depressed in some examples), and the housing 1040 is pushed in the distal direction.

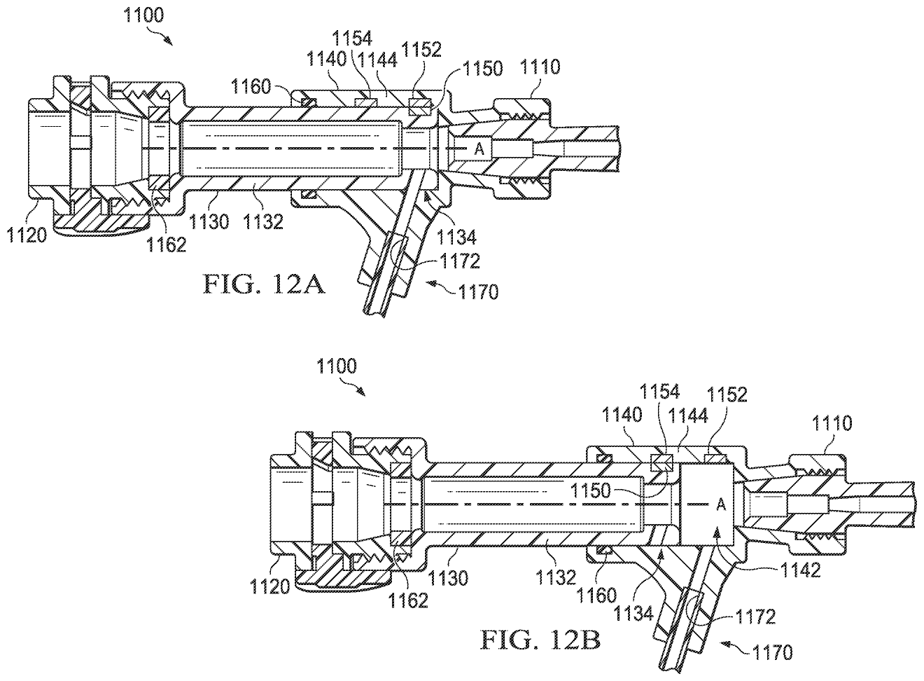

FIG. 12A provides a cross-sectional side view of an imaging coupler 1100 in a collapsed configuration, and FIG. 12B provides a cross-sectional side view of the imaging coupler 1100 in an extended configuration. As discussed above with respect to FIGS. 4A and 4B, the imaging coupler 1100 allows for an insertion distance of the imaging instrument 405 to be adjusted without moving the catheter 310. The imaging coupler 1100 includes an instrument connector 1110, a catheter connector 1120, a body portion 1130, a housing 1140 that is movable relative to the body portion 1130, and a magnet 1150. The magnet 1150 may maintain a position of the housing 1140 relative to the body portion 1130, which will be discussed in further detail below. The magnet 1150 may be part of the body portion 1130.

The housing 1140 may include a cavity 1142, and the body portion 1130 extends within the cavity 1142 and is movable within the cavity 1142 longitudinally along a longitudinal axis A. For example, a proximal end 1134 of the body portion 1130 may remain within the cavity 1142 when the imaging coupler 1100 is in the extended configuration.

As discussed above with respect to FIGS. 4A and 4B, the imaging coupler 1100 may couple to the imaging instrument 405 via the instrument connector 1110, and the imaging coupler 1100 may couple to the catheter 310 via the catheter connector 1120. As further discussed above, the body portion 1130 may couple to the catheter connector 1120. In some examples, the housing 1140 may couple to the instrument connector 1110. The imaging instrument 405 may extend along a longitudinal axis A of the body portion 1130 and may extend through the instrument connector 1110, through the housing 1140, through the body portion 1130, through the catheter connector 1120, and within the catheter 310 as previously described with respect to FIGS. 4A and 4B. The instrument connector 1110 may be pulled back from the body portion 1130 in a proximal direction that is parallel with the longitudinal axis A of the body portion 1130. When the instrument connector 1110 moves in the proximal direction, the distal end of the imaging instrument 405 is retracted within the lumen of the catheter 310. Additionally, the imaging coupler 1100 may also include a proximal seal 1160 and a distal seal 1162, which may be similar to the seals 460 and 462, respectively, as discussed above with respect to FIGS. 4A and 4B. The imaging coupler 1100 may also include a fluid connector 1170, which may be similar to the fluid connector 570 discussed above with respect to FIGS. 5A and 5B.

In some examples, when the magnet 1150 is aligned with a proximal magnet 1152 of the housing 1140, the imaging coupler 1100 may be in the collapsed configuration, and the magnet 1150 may maintain the imaging coupler 1100 in the collapsed configuration. When the magnet 1150 is aligned with a distal magnet 1154 of the housing 1140, the imaging coupler 1100 may be in the extended configuration, and the magnet 1150 may maintain the imaging coupler 1100 in the extended configuration. When the magnet 1150 is not aligned with the proximal magnet 1152 or the distal magnet 1154, the imaging coupler 1100 may be moved between the collapsed configuration and the extended configuration.

In some examples, the magnet 1150 may be embedded in a wall 1132 of the body portion 1130. The proximal magnet 1152 and the distal magnet 1154 may each be embedded in a wall 1144 of the housing 1140. The imaging coupler 1100 may include any number of additional magnets, which may be embedded within the wall 1132 and/or within the wall 1144.

When the proximal magnet 1152 is aligned with the magnet 1150, a magnetic force between the magnets 1152, 1150 may hold the imaging coupler 1100 in a locked configuration. In some examples, when the instrument connector 1110 is moved in a proximal direction, which may be done via an axial pulling force, the axial pulling force may overcome the magnetic force between the magnets 1152, 1150. When the axial pulling force overcomes the magnetic force, the housing 1140 is pulled in the proximal direction, and the magnet 1152 is pulled to a position proximal of the magnet 1150. As the housing 1140 continues to move in the proximal direction, the distal magnet 1154 will become aligned with the magnet 1150. When the distal magnet 1154 is aligned with the magnet 1150, a magnetic force between the magnets 1154, 1150 may hold the imaging coupler 1100 in the locked configuration. In some examples, when the instrument connector 1110 is moved in a distal direction, which may be done via an axial pushing force, the axial pushing force may overcome the magnetic force between the magnets 1154, 1150. When the axial pushing force overcomes the magnetic force, the housing 1140 is pushed in the distal direction, and the magnet 1154 is pushed to a position distal of the magnet 1150. In some examples, when the proximal magnet 1152 is aligned with the magnet 1150, the imaging coupler 1100 may be in a collapsed configuration. When the distal magnet 1154 is aligned with the magnet 1150, the imaging coupler 1100 may be in an extended configuration.

If the imaging coupler 1100 is in the locked position when fluid is supplied into the cavity 1142, the imaging coupler 1100 may remain in the locked position despite the pressure from the fluid. In this locked position, a magnetic force between the magnets 1150, 1152, for example, may prevent the housing 1140 from being pushed in the proximal direction by a fluid force imparted by the fluid onto the instrument connector 1110 to which the housing 1140 is coupled.

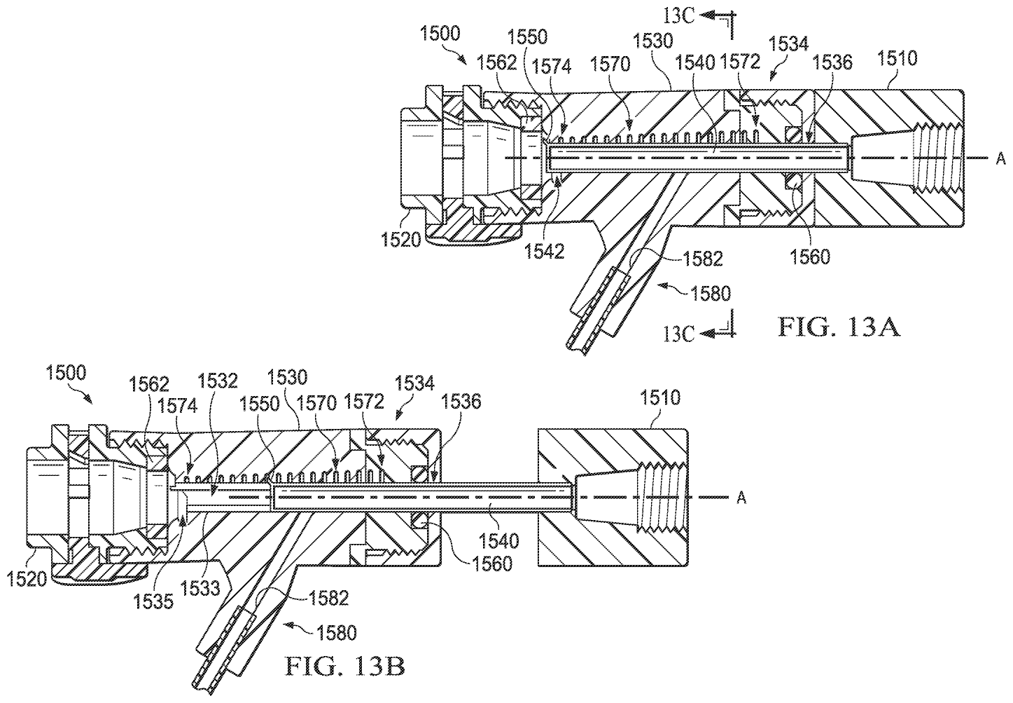

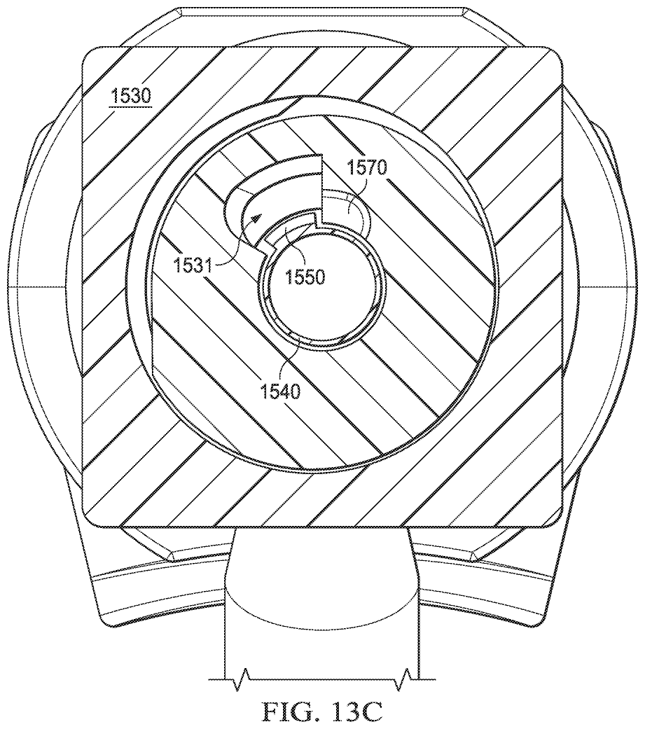

FIG. 13A provides a cross-sectional side view of an imaging coupler 1500 in a collapsed configuration, and FIG. 13B provides a cross-sectional side view of the imaging coupler 1500 in an extended configuration. As discussed above with respect to FIGS. 4A and 4B, the imaging coupler 1500 allows for an insertion distance of the imaging instrument 405 to be adjusted without moving the catheter 310. The imaging coupler 1500 includes an instrument connector 1510, a catheter connector 1520, a body portion 1530, a tubular member 1540 that is movable relative to the body portion 1530, and a tab 1550 at a distal end 1542 of the tubular member 1540. The tab 1550 may maintain a position of the tubular member 1540 relative to the body portion 1530, which will be discussed in further detail below. In some examples, the tubular member 1540 may include more than one tab 1550 (e.g., two tabs, three tabs, or any other number of tabs).

The body portion 1530 may include a cavity 1532, and the tubular member 1540 extends within the cavity 1532 and is movable within the cavity 1532 longitudinally along a longitudinal axis A. The cavity 1532 is defined by an inner surface 1533 of the body portion 1530, and the distal end 1542 of the tubular member 1540 may remain within the cavity 1532 when the imaging coupler 1500 is in the extended configuration. The body portion 1530 includes a proximal end 1534 with a proximal opening 1536. The tubular member 1540 extends through the proximal opening 1536. In some examples, an outer diameter of the tab 1550 is greater than an outer diameter of the proximal opening 1536. This may help prevent the tubular member 1540 from being pulled completely out of the body portion 1530. The tab 1550 may be flared or tapered. The body portion 1530 includes a distal lumen 1535, which is defined by the inner surface 1533 of the body portion 1530. The distal lumen 1535 may be sized to receive the tab 1550 of the tubular member 1540.

As discussed above with respect to FIGS. 4A and 4B, the imaging coupler 1500 may couple to the imaging instrument 405 via the instrument connector 1510, and the imaging coupler 1500 may couple to the catheter 310 via the catheter connector 1520. As further discussed above, the body portion 1530 may extend between and couple to the instrument connector 1510 and the catheter connector 1520. The imaging instrument 405 may extend along the longitudinal axis A of the tubular member 1540 and may extend through the instrument connector 1510, through the tubular member 1540, through the catheter connector 1520, and within the catheter 310 as previously described with respect to FIGS. 4A and 4B. In some examples, the instrument connector 1510 may be pulled back from the body portion 1530 in a proximal direction that is parallel with the longitudinal axis A of the tubular member 1540. When the instrument connector 1510 moves in the proximal direction, the distal end of the imaging instrument 405 is retracted within the lumen of the catheter 310. Additionally, the imaging coupler 1500 may also include a proximal seal 1560 and a distal seal 1562, which may be similar to the seals 460 and 462, respectively, as discussed above with respect to FIGS. 4A and 4B.

As seen in FIG. 13A, the body portion 1530 may include one or more projections 1570. The projections 1570 may extend from the inner surface 1533 into the cavity 1532. The tab 1550 may be sized and shaped to fit between two of the projections 1570. The tubular member 1540 is rotatable around its longitudinal axis A. In some examples, the tubular member 1540 may be rotated until the tab 1550 is not aligned with the projections 1570. In some examples, when the tab 1550 is not aligned with the projections 1570, the tab 1550 is positioned within an open channel 1531 in the cavity 1532. When the tab 1550 is not aligned with the projections 1570, the tubular member 1540 may be moved in a proximal and/or distal direction within the cavity 1532, and the imaging coupler 1500 is in an unlocked configuration. When the tab 1550 is aligned with the projections 1570 (e.g., positioned between two projections 1570), the tubular member 1540 may not be moved in a proximal and/or distal direction within the cavity 1532, and the imaging coupler 1500 is in a locked configuration.

In some examples, each of the projections 1570 may be the same size. In other examples, the projections 1570 may vary in size (e.g., in radial length, thickness, or both). For example, a proximal most projection 1572 may be the largest projection, and the remaining projections 1570 may gradually decrease in size where a distal most projection 1574 is the smallest projection, as shown in FIGS. 13A and 13B. In other examples, the proximal most projection 1572 may be the smallest projection, and the remaining projections 1570 may gradually increase in size where the distal most projection 1574 is the largest projection. In other examples, the size of the projections 1570 may alternate between large and small, vary randomly, or vary in any other pattern.

The tubular member 1540 may be rotated so that the tab 1550 is positioned between any two projections of the projections 1570. This allows for the tubular member 1540 to be axially locked at one or more positions between the proximal most projection 1572 of the projections 1570 and the distal most projection 1574 of the projections 1570. In such examples, the imaging coupler 1500 may be axially locked at one or more positions between the collapsed configuration and the extended configuration.

FIG. 13C provides a cross-sectional view of the imaging coupler 1500 in the unlocked configuration. The cross-sectional view is provided from a perspective looking in the distal direction. In some examples, the imaging coupler 1500 is in the unlocked position when the tab 1550 is not aligned with the projections 1570. For example, FIG. 13C shows the tab 1550 not aligned with the projections 1570. The tubular member 1540 is rotated such that the tab 1550 is not aligned with the projections 1570. When the imaging coupler 1500 is in the unlocked configuration, the tubular member 1540 may be moved in the proximal direction or the distal direction because the tab 1550 may move past the projections 1570.

In some examples, the imaging coupler 1500 may also include a biasing member (not shown), such as a spring or any other suitable biasing member similar to the biasing member 450 in FIGS. 4A and 4B. The biasing member may be coupled to the tubular member 1540. In some examples, the biasing member may surround the tubular member 1540. The biasing member may bias the tubular member 1540 in the distal direction, which may bias the imaging coupler 1500 toward the collapsed configuration. Alternatively, the biasing member may bias the tubular member 1540 in the proximal direction, which may bias the imaging coupler 1500 in the extended configuration.

The imaging coupler 1500 may also include a fluid connector 1580, which may include tubing 1582 (e.g., the tubing 254). In some examples, the fluid supply system 250 includes the fluid connector 570. Cleaning fluid may be supplied from the fluid delivery system 256, through the tubing 1582 of the fluid connector 1580, and into the cavity 1532 of the body portion 1530. The cleaning fluid may then be supplied to a lens of the imaging instrument 405 and/or to a lens of a camera of the imaging instrument 405 to clean the lens, for example, and remove any visual obstructions on the lens, such as blood, mucus, and/or any other fluids/particles. When the fluid is supplied into the cavity 1532, the seals 1560, 1562 prevent the fluid from leaking out of the imaging coupler 1500. If the imaging coupler 1500 is in the locked position when fluid is supplied into the cavity 1532, the imaging coupler 1500 may remain in the locked position, despite the pressure from the fluid. In this locked position, the tab 1550 and one or more of the projections 1570 may prevent the tubular member 1540 from being pushed in a proximal direction by a fluid force imparted by the fluid onto the instrument connector 1510 to which the tubular member 1540 is coupled.

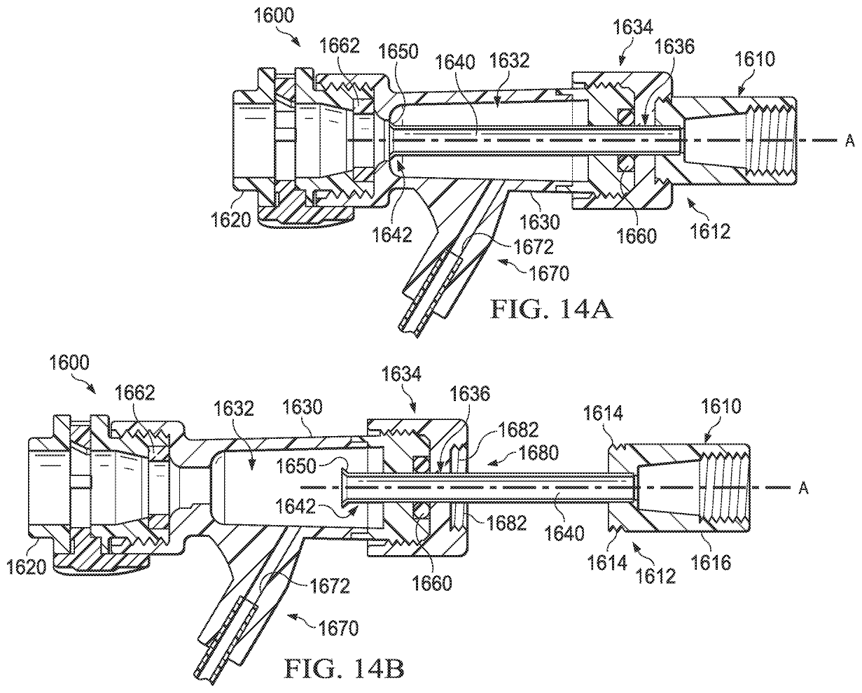

FIG. 14A provides a cross-sectional side view of an imaging coupler 1600 in a collapsed configuration, and FIG. 14B provides a cross-sectional side view of the imaging coupler 1600 in an extended configuration. As discussed above with respect to FIGS. 4A and 4B, the imaging coupler 1600 allows for an insertion distance of the imaging instrument 405 to be adjusted without moving the catheter 310. The imaging coupler 1600 includes an instrument connector 1610, a catheter connector 1620, a body portion 1630, and a tubular member 1640 that is movable relative to the body portion 1630. The instrument connector 1610 may be engaged with the body portion 1630 via a threaded connection (e.g., via threads), which will be discussed in further detail below.

The body portion 1630 may include a cavity 1632, and the tubular member 1640 extends within the cavity 1632 and is movable within the cavity 1632 longitudinally along a longitudinal axis A. In some examples, a distal end 1642 of the tubular member 1640 may remain within the cavity 1632 when the imaging coupler 1600 is in the extended configuration. The body portion 1630 also includes a proximal end 1634 with a proximal opening 1636. The tubular member 1640 extends through the proximal opening 1636. In some examples, the tubular member 1640 includes a ring 1650 at the distal end 1642 of the tubular member 1640. In some examples, an outer diameter of the ring 1650 is greater than an outer diameter of the proximal opening 1636 of the body portion 1630. This may help prevent the tubular member 1640 from being pulled completely out of the body portion 1630.

As discussed above with respect to FIGS. 4A and 4B, the imaging coupler 1600 may couple to the imaging instrument 405 via the instrument connector 1610, and the imaging coupler 1600 may couple to the catheter 310 via the catheter connector 1620. As further discussed above, the body portion 1630 may extend between and couple to the instrument connector 1610 and the catheter connector 1620. The imaging instrument 405 may extend along the longitudinal axis A of the tubular member 1640 and may extend through the instrument connector 1610, through the tubular member 1640, through the catheter connector 1620, and within the catheter 310 as previously described with respect to FIGS. 4A and 4B. In some examples, the instrument connector 1610 may be pulled back from the body portion 1630 in a proximal direction that is parallel with the longitudinal axis A of the tubular member 1640. When the instrument connector 1610 moves in the proximal direction, the distal end of the imaging instrument 405 is retracted within the lumen of the catheter 310. Additionally, the imaging coupler 1600 may also include a proximal seal 1660 and a distal seal 1662, which may be similar to the seals 460 and 462, respectively, as discussed above with respect to FIGS. 4A and 4B. The imaging coupler 1600 may also include a fluid connector 1670, which may be similar to the fluid connector 570 discussed above with respect to FIGS. 5A and 5B.

In some examples, the instrument connector 1610 may be threadedly engageable with the body portion 1630. This threaded engagement may maintain a position of the tubular member 1640 relative to the body portion 1630, as will be discussed in more detail below. In some examples, as shown in FIG. 14A, when the imaging coupler 1600 is in the collapsed configuration, the instrument connector 1610 is engaged with the body portion 1630 via a threaded connection (e.g., via threads). In other examples, as shown in FIG. 14B, when the imaging coupler 1600 is in the extended configuration, the instrument connector 1610 is disengaged from the body portion 1630.

As shown in FIG. 14B, the proximal end 1634 of the body portion 1630 may include a recess 1680. A distal end 1612 of the instrument connector 1610. may be sized and shaped to mate with and/or be received by the recess 1680. In some examples, an inner surface of the recess 1680 may include one or more grooves 1682. The grooves 1682 may be sized and shaped to receive one or more threads 1614, which may be on an outer surface 1616 of the instrument connector 1610. In alternative examples, the outer surface 1616 of the instrument connector 1610 may include one or more grooves, and the inner surface of the recess 1680 may include one or more corresponding threads.

In some examples, the instrument connector 1610 may be rotated relative to the body portion 1630. The instrument connector 1610 may be rotated while the body portion 1630 remains rotationally stationary. For example, the instrument connector 1610 may be rotated in a counterclockwise manner about the axis A of the tubular member 1640. This counterclockwise rotation moves the instrument connector 1610 in the proximal direction, which disengages the distal end 1612 of the instrument connector 1610 from the recess 1680 of the body portion 1630. When the instrument connector 1610 is disengaged from the body portion 1630, the instrument connector 1610 and the tubular member 1640 may be free to move along the longitudinal axis A relative to and independent of the body portion 1630.

The instrument connector 1610 may be rotated in a clockwise manner about the axis A to move the instrument connector 1610 in the distal direction. This clockwise rotation engages the distal end 1612 of the instrument connector 1610 with the recess 1680 of the body portion 1630. When the instrument connector 1610 is engaged (e.g., threadedly engaged) with the body portion 1630, the instrument connector 1610 and the tubular member 1640 may be coupled to and axially fixed relative to the body portion 1630. In some examples, when the instrument connector 1610 is engaged with the body portion 1630, the imaging coupler 1600 is in the collapsed configuration.

In alternative examples, the instrument connector 1610 may be rotated in a clockwise manner about the axis A of the tubular member 1640 to move the instrument connector 1610 in the proximal direction, which disengages the distal end 1612 of the instrument connector 1610 from the recess 1680 of the body portion 1630. In such examples, the instrument connector 1610 may be rotated in a counterclockwise manner about the axis A to move the instrument connector 1610 in the distal direction, which engages the distal end 1612 of the instrument connector 1610 with the recess 1680 of the body portion 1630.

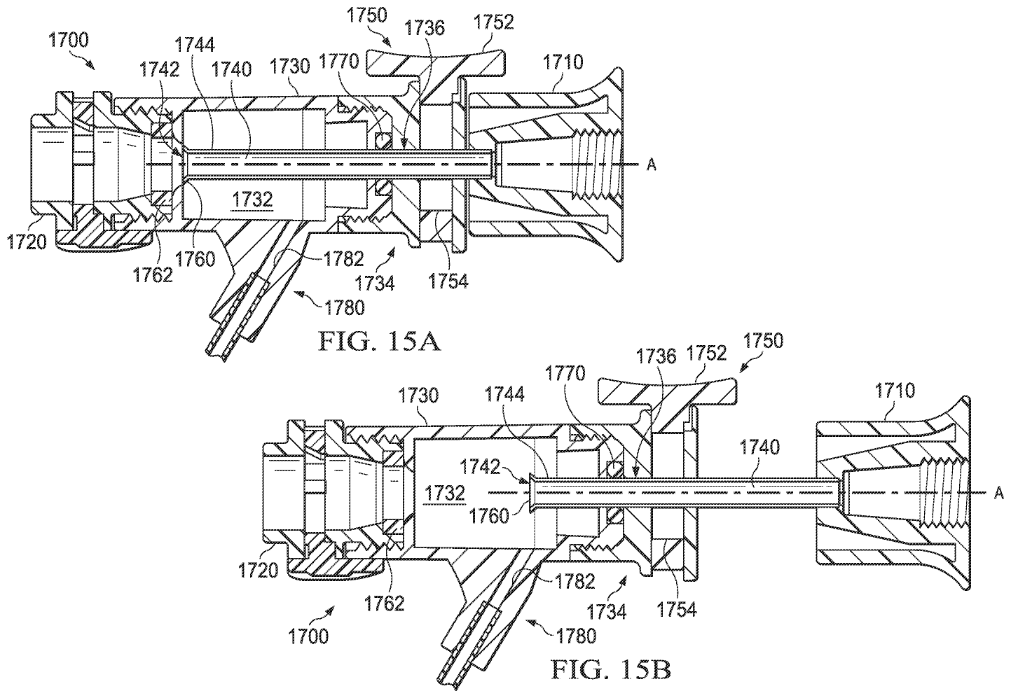

FIG. 15A provides a cross-sectional side view of an imaging coupler 1700 in a collapsed configuration, and FIG. 15B provides a cross-sectional side view of the imaging coupler 1700 in an extended configuration. As discussed above with respect to FIGS. 4A and 4B, the imaging coupler 1700 allows for an insertion distance of the imaging instrument 405 to be adjusted without moving the catheter 310. The imaging coupler 1700 includes an instrument connector 1710, a catheter connector 1720, a body portion 1730, a tubular member 1740 that is movable relative to the body portion 1730, and a lock assembly 1750. The lock assembly 1750 may maintain a position of the tubular member 1740 relative to the body portion 1730, which will be discussed in further detail below.

The body portion 1730 may include a cavity 1732, and the tubular member 1740 extends within the cavity 1732 and is movable within the cavity 1732 longitudinally along a longitudinal axis A. In some examples, a distal end 1742 of the tubular member 1740 may remain within the cavity 1732 when the imaging coupler 1700 is in the extended configuration. The body portion 1730 also includes a proximal end 1734 with a proximal opening 1736. The tubular member 1740 extends through the proximal opening 1736. In some examples, the tubular member 1740 includes a ring 1760 at the distal end 1742 of the tubular member 1740. In some examples, an outer diameter of the ring 1760 is greater than an outer diameter of the proximal opening 1736 of the body portion 1730. This may help prevent the tubular member 1740 from being pulled completely out of the body portion 1730.

As discussed above with respect to FIGS. 4A and 4B, the imaging coupler 1700 may couple to the imaging instrument 405 via the instrument connector 1710, and the imaging coupler 1700 may couple to the catheter 310 via the catheter connector 1720. As further discussed above, the body portion 1730 may extend between and couple to the instrument connector 1710 and the catheter connector 1720. The imaging instrument 405 may extend along the longitudinal axis A of the tubular member 1740 and may extend through the instrument connector 1710, through the tubular member 1740, through the catheter connector 1720, and within the catheter 310 as previously described with respect to FIGS. 4A and 4B. In some examples, the instrument connector 1710 may be pulled back from the body portion 1730 in a proximal direction that is parallel with the longitudinal axis A of the tubular member 1740. When the instrument connector 1710 moves in the proximal direction, the distal end of the imaging instrument 405 is retracted within the lumen of the catheter 310. Additionally, the imaging coupler 1700 may also include a proximal seal 1770 and a distal seal 1772, which may be similar to the seals 460 and 462, respectively, as discussed above with respect to FIGS. 4A and 4B. The imaging coupler 1700 may also include a fluid connector 1780, which may be similar to the fluid connector 570 discussed above with respect to FIGS. 5A and 5B.

The lock assembly 1750 may be coupled to the body portion 1730. In other examples, the lock assembly 1750 may be integrally formed with the body portion 1730. A tab 1752 of the lock assembly 1750 may be depressed in a direction that is radially inward toward the axis A of the tubular member 1740. When the tab 1752 is depressed, a key 1754 of the lock assembly 1750 may be moved radially outward. In some examples, the tab 1752 may include an internal biasing member (not shown) that biases the tab 1752 in a radially outward direction. For example, when the tab 1752 is not depressed, the tab 1752 may remain in an extended position due to a biasing force imparted by the internal biasing member.

In some examples, the lock assembly 1750 may be movable between an engaged configuration and a disengaged configuration. In the engaged configuration, the tab 1752 is not depressed, and the key 1754 is engaged with an outer surface 1744 of the tubular member 1740. When the lock assembly 1750 is in the engaged configuration, the tubular member 1740 is not movable in the proximal direction. Thus, when the lock assembly 1750 is in the engaged configuration, the imaging coupler 1700 is in a locked configuration. When the lock assembly 1750 is in the disengaged configuration, the tab 1752 is depressed, and the key 1754 is disengaged from the outer surface 1744 of the tubular member 1740. When the lock assembly 1750 is in the disengaged configuration, the tubular member 1740 is movable in the proximal direction. Thus, when the lock assembly 1750 is in the disengaged configuration, the imaging coupler 1700 is in an unlocked configuration. To move the tubular member 1740 in the proximal and/or distal direction the tab 1752 needs to be depressed, and the instrument connector 1710 is pulled in the proximal direction and/or is pushed in the distal direction.

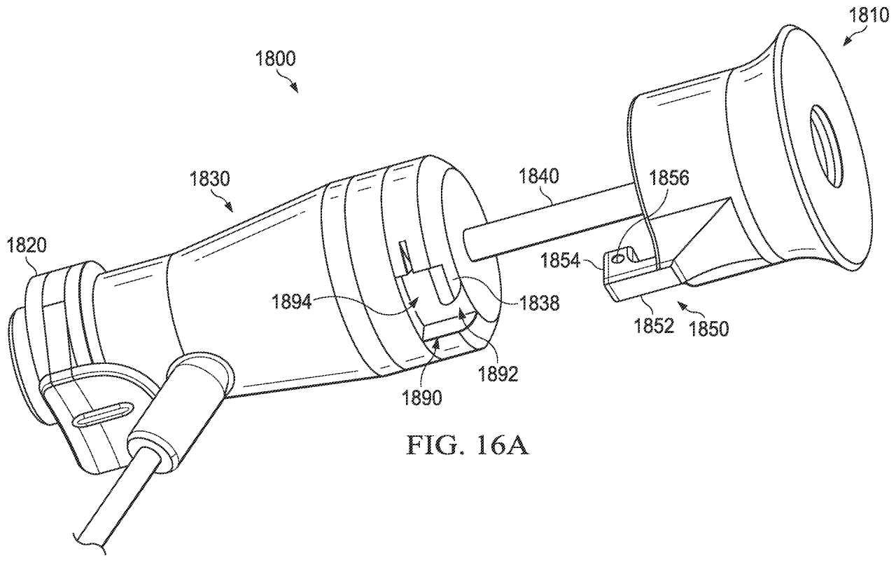

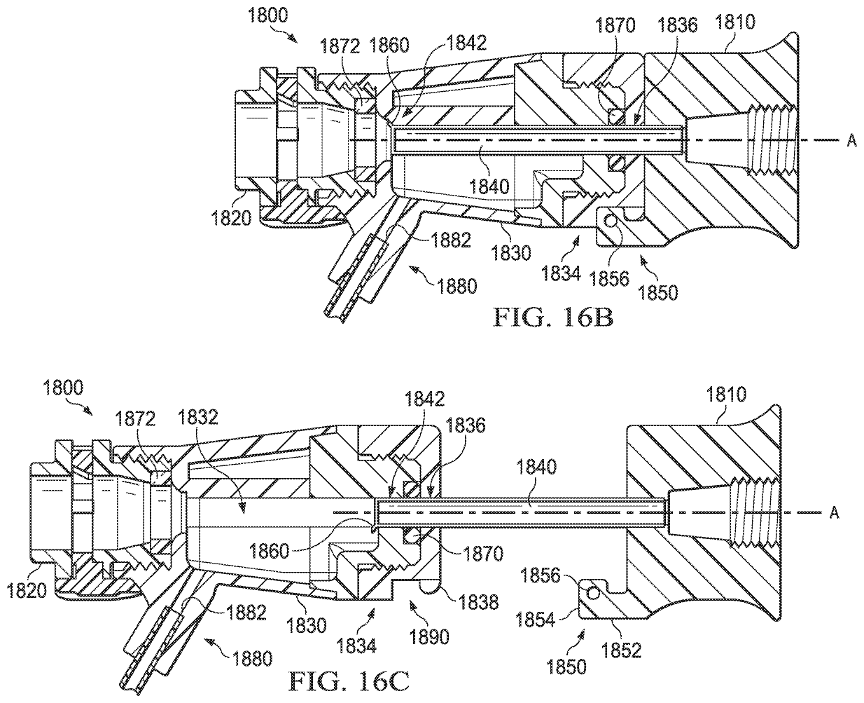

FIG. 16A illustrates an imaging coupler 1800 in an extended configuration. FIG. 16B provides a cross-sectional side view of the imaging coupler 1800 in a collapsed configuration, and FIG. 16C provides a cross-sectional side view of the imaging coupler 1800 in the extended configuration. As discussed above with respect to FIGS. 4A and 4B, the imaging coupler 1800 allows for an insertion distance of the imaging instrument 405 to be adjusted without moving the catheter 310. The imaging coupler 1800 includes an instrument connector 1810, a catheter connector 1820, a body portion 1830, and a tubular member 1840 that is movable relative to the body portion 1830. The instrument connector 1810 may be releasably engageable with the body portion 1830 by a locking member 1850, which will be discussed in further detail below.

As shown in FIGS. 16B and 16C, the body portion 1830 may include a cavity 1832, and the tubular member 1840 extends within the cavity 1832 and is movable within the cavity 1832 longitudinally along a longitudinal axis A. In some examples, a distal end 1842 of the tubular member 1840 may remain within the cavity 1832 when the imaging coupler 1800 is in the extended configuration. The body portion 1830 also includes a proximal end 1834 with a proximal opening 1836. The tubular member 1840 extends through the proximal opening 1836. In some examples, the tubular member 1840 includes a ring 1860 at the distal end 1842 of the tubular member 1840. In some examples, an outer diameter of the ring 1860 is greater than an outer diameter of the proximal opening 1836 of the body portion 1830. This may help prevent the tubular member 1840 from being pulled completely out of the body portion 1830.

As discussed above with respect to FIGS. 4A and 4B, the imaging coupler 1800 may couple to the imaging instrument 405 via the instrument connector 1810, and the imaging coupler 1800 may couple to the catheter 310 via the catheter connector 1820. As further discussed above, the body portion 1830 may extend between and couple to the instrument connector 1810 and the catheter connector 1820. The imaging instrument 405 may extend along the longitudinal axis A of the tubular member 1840 and may extend through the instrument connector 1810, through the tubular member 1840, through the catheter connector 1820, and within the catheter 310 as previously described with respect to FIGS. 4A and 4B. In some examples, the instrument connector 1810 may be pulled back from the body portion 1830 in a proximal direction that is parallel with the longitudinal axis A of the tubular member 1840. When the instrument connector 1810 moves in the proximal direction, the distal end of the imaging instrument 405 is retracted within the lumen of the catheter 310. Additionally, the imaging coupler 1800 may also include a proximal seal 1870 and a distal seal 1872, which may be similar to the seals 460 and 462, respectively, as discussed above with respect to FIGS. 4A and 4B. The imaging coupler 1800 may also include a fluid connector 1880, which may be similar to the fluid connector 570 discussed above with respect to FIGS. 5A and 5B.

In some examples, the instrument connector 1810 may be releasably engageable with the body portion 1830. This releasable engagement may maintain a position of the tubular member 1840 relative to the body portion 1830, as will be discussed in more detail below. In some examples, as shown in FIG. 16B, when the imaging coupler 1800 is in the collapsed configuration, the instrument connector 1810 is engaged with (e.g., coupled to, locked to, etc.) the body portion 1830. In other examples, as shown in FIG. 16C, when the imaging coupler 1800 is in the extended configuration, the instrument connector 1810 is disengaged from the body portion 1830.

As shown in FIGS. 16A and 16C, the proximal end 1834 of the body portion 1830 may include a recess 1890 (e.g., a groove, a notch, or any other recess) in a wall of the body portion 1830. The locking member 1850 of the instrument connector 1810 may be sized and shaped to mate with and/or be received by the recess 1890. In some examples, the locking member 1850 includes an elongate portion 1852 and a hook portion 1854. The hook portion 1854 may extend from the elongate portion 1852 in a radial direction towards the tubular member 1840. In some examples, the recess 1890 includes an entry recess 1892 and a locking recess 1894. A portion 1838 of the wall of the body portion 1830 may be adjacent to the locking recess 1894.

In some examples, the elongate portion 1852 and the hook portion 1854 are sized and shaped to be received by the entry recess 1892. When the hook portion 1854 is fully received within the entry recess 1892, the instrument connector 1810 may be rotated relative to the body portion 1830. The instrument connector 1810 may be rotated while the body portion 1830 remains rotationally stationary. For example, the instrument connector 1810 may be rotated in a clockwise manner about the axis A of the tubular member 1840. This clockwise rotation moves the hook portion 1854 into the locking recess 1894, which engages the instrument connector 1810 with the body portion 1830. When the instrument connector 1810 is engaged with the body portion 1830, the instrument connector 1810 and the tubular member 1840 may be coupled to and axially fixed relative to the body portion 1830. In some examples, when the instrument connector 1810 is engaged with the body portion 1830, the imaging coupler 1800 is in the collapsed configuration.

In some examples, the hook portion 1854 may include a coupling member 1856 (e.g., a magnet, latch, or other similar coupling member). The coupling member 1856 may be embedded within the hook portion 1854 and/or may be coupled to an outside surface of the hook portion 1854. The locking recess 1894 may include a corresponding coupling member (e.g., a magnet, hook, or other similar coupling member) that may mate with the coupling member 1856 of the hook portion 1854. This mating connection may hold the hook portion 1854 in the locking recess 1894.

The hook portion 1854 may be held in the locking recess 1894 until the instrument connector 1810 is rotated in a counterclockwise manner, for example, about the axis A of the tubular member 1840. This counterclockwise rotation moves the hook portion 1854 out of the locking recess 1894 and into the entry recess 1892. When the hook portion 1854 is in the entry recess 1892, the instrument connector 1810 may be free to move in a proximal direction along the longitudinal axis A relative to and independent of the body portion 1630.

In alternative examples, the hook portion 1854 may be held in the locking recess 1894 until the instrument connector 1810 is rotated in a clockwise manner, for example, about the axis A of the tubular member 1840. This clockwise rotation moves the hook portion 1854 out of the locking recess 1894 and into the entry recess 1892.

During a medical procedure, the imaging instrument (e.g., the imaging instrument 130, 210, and 405) may be temporarily or permanently removed from the catheter 310 to make way for other medical tools, such as a biopsy tool or treatment tool. To prevent damage to the imaging instrument, to prevent coiling/entanglement of the imaging instrument, to fully contain the imaging instrument, and/or for convenience, a storage device may be provided to stow the imaging instrument. While the storage device will be described below as storing an imaging instrument, it will be understood that the storage device may be used to store other instruments, such as biopsy instruments, therapeutic instruments, etc. For example, one instrument (e.g., a biopsy instrument) may be stored in the storage device while another instrument (e.g., an imaging instrument) is positioned in the catheter 310. When the imaging instrument is removed from the catheter 310, the imaging instrument may be stored in the storage device, and the biopsy instrument may be inserted into the catheter 310. In some examples, multiple instruments may be simultaneously stored in the storage device.

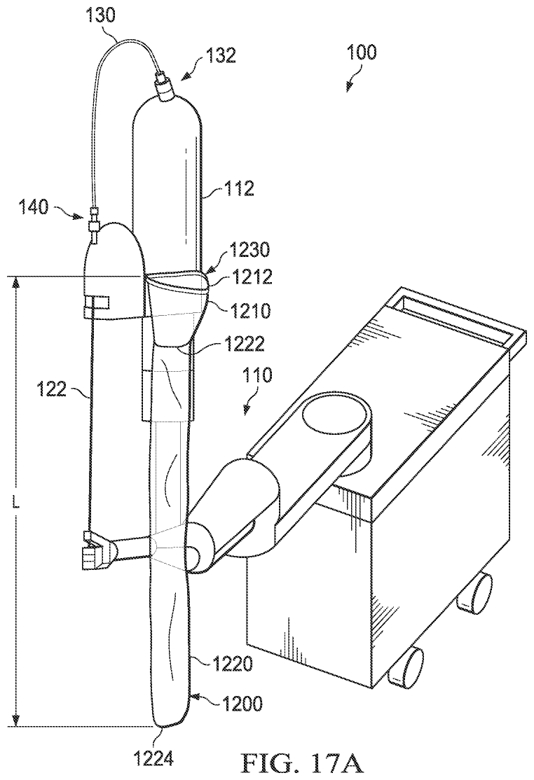

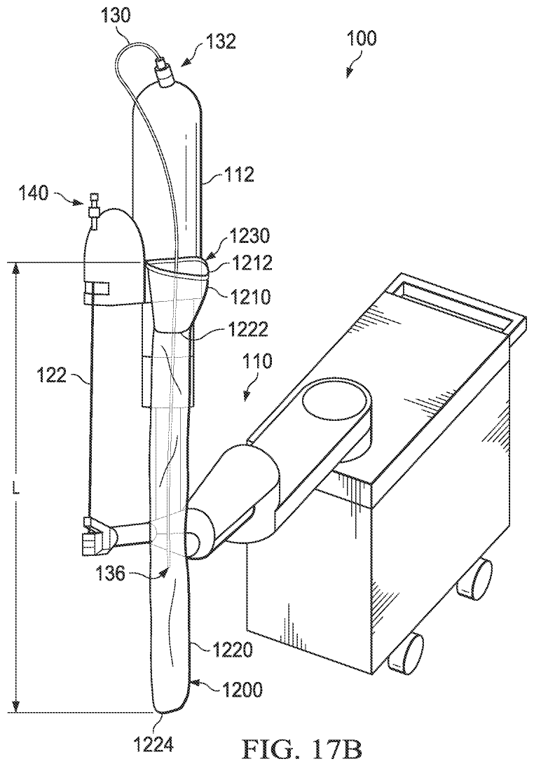

FIG. 17A provides a perspective view of a storage device 1200, which may receive the imaging instrument 130. The storage device 1200 may be removably coupled to the manipulator arm 112. In some examples, the imaging instrument 130 may be decoupled from the imaging coupler 140, which may be any one of the imaging couplers discussed above. In some examples, the imaging instrument 130 may remain coupled to the imaging coupler 140, and together the imaging instrument 130 and imaging coupler 140 may be decoupled from a body portion of the catheter 122. The imaging instrument 130 may also be removed from the catheter 122. In some examples, the imaging instrument 130 may be removed from the catheter 122 so that a different instrument (e.g., a biopsy instrument, an ultrasound instrument, or an electromagnetic instrument) can be placed within the catheter 122. For example, after the medical instrument 120 has been navigated to a target location within an anatomy, the imaging instrument 130 may be removed from the catheter 122, and a biopsy instrument may be inserted into the catheter 122. After a biopsy is performed, the medical instrument 120 may be navigated to another target location. To navigate the medical instrument 120 to the other target location, the biopsy instrument may be removed from the catheter 122, and the imaging instrument 130 may be reinserted into the catheter 122. When the imaging instrument 130 is removed from the catheter 122, the proximal portion 132 of the imaging instrument 130 may remain coupled to the manipulator arm 112 of the manipulator assembly 110, and a distal end 136 (see FIG. 17B) of the imaging instrument 130 may be placed into the storage device 1200.

In some examples, the storage device 1200 may be a flexible container, such as a plastic bag. In some examples, the storage device 1200 may be a tube, such as a plastic tube, which may have an open end or a closed end. In some examples, the tube may be rigid (e.g., a PVC tube). In other examples, the tube may be flexible and/or bendable. Additionally or alternatively, the storage device 1200 may be a bag, a tube, or the like, that may hang on a hook coupled to the manipulator arm 112. Additionally or alternatively, the storage device 1200 may be an elongate foam member that includes a slot in which the imaging instrument 130 may be pressed and retained. Additionally or alternatively, the storage device 1200 may be an elongate “J-shaped” or “U-shaped” tube within which the imaging instrument 130 may be inserted. In such embodiments, a distal portion of the storage device 1200 is curved, and a distal portion of the imaging instrument 130 would be curved when inserted within the storage device 1200. The storage device 1200 may be any other suitable storage device.

In some examples, the storage device 1200 may be sterilizable and therefore may be able to withstand a sterilization process, which may be performed by an autoclave, for example. The storage device 1200 may be able to withstand any other sterilization process. In some examples, the storage device 1200 may be single use and may be disposed of after completion of a medical procedure using the imaging instrument 130.

As seen in FIG. 17A, the storage device 1200 includes a proximal portion 1210 coupled to an elongate portion 1220. The proximal portion 1210 includes a rim 1212 that defines an opening 1230. In some examples, the opening 1230 has a first perimeter, which may be 25 cm, but may be any other length. The elongate portion 1220 has a second perimeter, which may be 10 cm, but may be any other length. The proximal portion 1210 may be tapered in an axial direction from the first perimeter to the second perimeter such that the proximal portion 1210 forms a funnel extending from a widest point at the opening 1230 to a narrowest point at a proximal end 1222 of the elongate portion 1220. The proximal portion 1210 may be shaped like a funnel to allow for ease of insertion when the imaging instrument 130 is placed through the opening 1230. The wide opening 1230 may provide the user with a larger target area within which to introduce the distal end 136 of the imaging instrument 130. This may facilitate a quicker transition between instruments when the imaging instrument 130 is switched out for another instrument within the catheter 122. The wide opening 1230 may also help ensure the user does not miss the opening 1230 when trying to place the imaging instrument 130 into the storage device 1200.

The storage device 1200 includes a length L. In some examples, the length L is long enough that when the imaging instrument 130 is fully inserted in the storage device 1200, as shown in FIG. 17B, a distal end 136 of the imaging instrument 130 is proximal of a distal end 1224 of the storage device 1200. In such examples, the imaging instrument 130 may be in an unbent configuration when inserted in the storage device 1200. Additionally, the length L may be short enough that the distal end 1224 does not touch the floor of the operating room or the floor of any other room where the medical system 100 is located. For example, the length L may be short enough that the distal end 1224 does not touch the floor when the manipulator arm 112 is at its highest position (e.g., corresponding to full retraction of the catheter 122) and/or when the manipulator arm 112 is at its lowest position (e.g., corresponding to full insertion of the catheter 122 into patient anatomy). In some examples, the length L may be approximately 40 inches but may be any other suitable length that ensures the distal end 136 of the imaging instrument 130 does not reach the distal end 1224. In some examples, the distal end 1224 is sealed. This may help prevent any fluids that may be present on the imaging instrument 130 from dripping onto the floor of the operating room.

In some examples, the elongate portion 1220 may have a generally tubular shape but may have any other shape in other examples. The tubular shape may provide a space-saving profile, which may help prevent the storage device 1200 from interfering with the surgeon during the medical procedure. For example, the tubular shape may help ensure that the elongate portion 1220 remains positioned close to the manipulator arm 112 so the storage device 1200 does not disturb or impede an operator during the medical procedure.

In some examples, the funnel shape of the proximal portion 1210 may help prevent the imaging instrument 130 from folding/curling back in on itself as the distal end 136 of the imaging instrument 130 passes through the proximal portion 1210. In such examples, the distal end 136 may remain pointed toward the distal end 1224 of the elongate portion 1220 as the imaging instrument 130 is inserted into the storage device 1200.

In some cases, the storage device 1200 includes one channel or compartment defined by an inner surface of the storage device 1200. The one channel can receive the imaging instrument 130 and/or any other instruments (e.g., a biopsy instrument, an ultrasound instrument, or an electromagnetic instrument) at the same time. In alternative examples, the storage device 1200 includes multiple channels or compartments, such as two channels, three channels, four channels, or any other number of channels. When multiple instruments are inserted into the storage device 1200 at the same time, each instrument may be inserted into its own individual channel. This keeps the instruments separated while they are in the storage device 1200, which can help prevent the instruments from tangling, bumping against each other, or otherwise interfering with each other. In some examples, the channels are sized and shaped based on the size and/or shape of the instrument designated to be inserted into each respective channel. Thus, the channels may be different sizes and/or shapes. Alternatively, the channels may all be the same size and shape. The channels may be defined by dividers. In some examples, the dividers extend the full length of the storage device 1200 from the opening 1230 to the distal end 1224. In other examples, the dividers start at the proximal end 1222 of the elongate portion 1220 and extend to the distal end 1224.

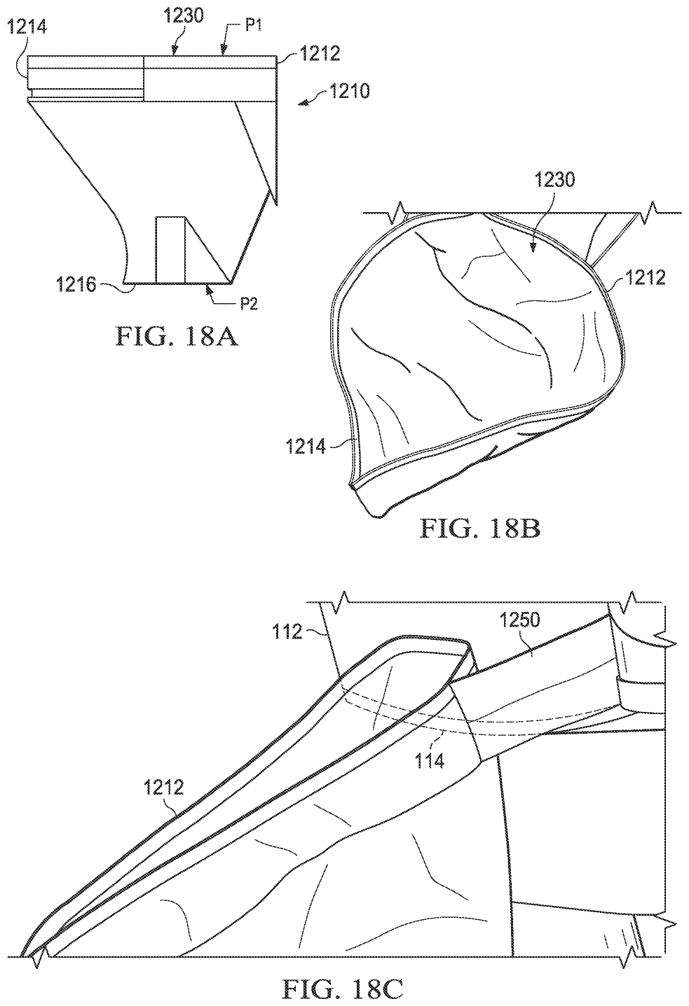

FIGS. 18A-18C provide various views of the proximal portion 1210. For example, FIG. 18A provides a side view of the proximal portion 1210 (e.g., when the storage device 1200 is in a folded configuration). As discussed above, the proximal portion 1210 forms a funnel extending from a widest point at the opening 1230 to a narrowest point at a proximal end 1222 of the elongate portion 1220, which may coincide with a distal end 1216 of the proximal portion 1210. The widest point may be defined by the first perimeter P1 of the opening 1230, and the narrowest point may be defined by the second perimeter P2 of the distal end 1216.

In some examples, the rim 1212 of the proximal portion 1210 may include a cuff 1214. The cuff 1214 may fold over a wire (not shown) so that the wire remains concealed by the cuff 1214. Concealing the wire in the cuff 1214 may prevent the user's clothing, such as gloves, gown, scrubs, or other clothing/equipment, from getting caught on the wire and/or being torn by the wire. In some examples, the wire is a flexible metal wire. In other examples, the wire may be a flexible plastic wire or any other flexible, bendable material. For example, as seen in FIG. 18B, the shape of the wire may be adjusted by the user to shape the rim 1212 to a shape desired by the user. The desired shape may be determined based on a variety of factors including, for example, the location of the manipulator assembly 110 in the operating room, the location of the surgeon relative to the storage device 1200, or the surgeon's height. Being able to adjust the shape of the opening 1230 by adjusting the shape of the wire may allow for the imaging instrument 130 to be more easily inserted into the storage device 1200.

As shown in FIG. 18C, the storage device 1200 also includes an attachment member 1250. In some examples, the attachment member 1250 may be a strap that fits over the manipulator arm 112. The attachment member 1250 may be transparent so one or more system indicators, such as the indicator 114 (e.g., a light or other indicator) on the manipulator arm 112, may be seen through the attachment member 1250. In some examples, the entire storage device 1200 is transparent so that one or more other indicators of the manipulator assembly 110 are not obscured by the storage device 1200. Additionally or alternatively, the attachment member 1250 may be a magnetic connector where a magnet in the rim 1212, for example, couples to a magnet in the manipulator arm 112. In alternative examples, the attachment member 1250 may be an adhesive connector that adhesively couples to the manipulator arm 112. In other examples, the manipulator arm 112 may include a hook, and the attachment member 1250 may hang on the hook. The storage device 1200 may be attached to the manipulator assembly 110 in any other suitable manner.

In some examples, the components discussed above may be part of a robotic-assisted system as described in further detail below. The robotic-assisted system may be suitable for use in, for example, surgical, robotic-assisted surgical, diagnostic, therapeutic, or biopsy procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general robotic-assisted, or robotic medical systems.

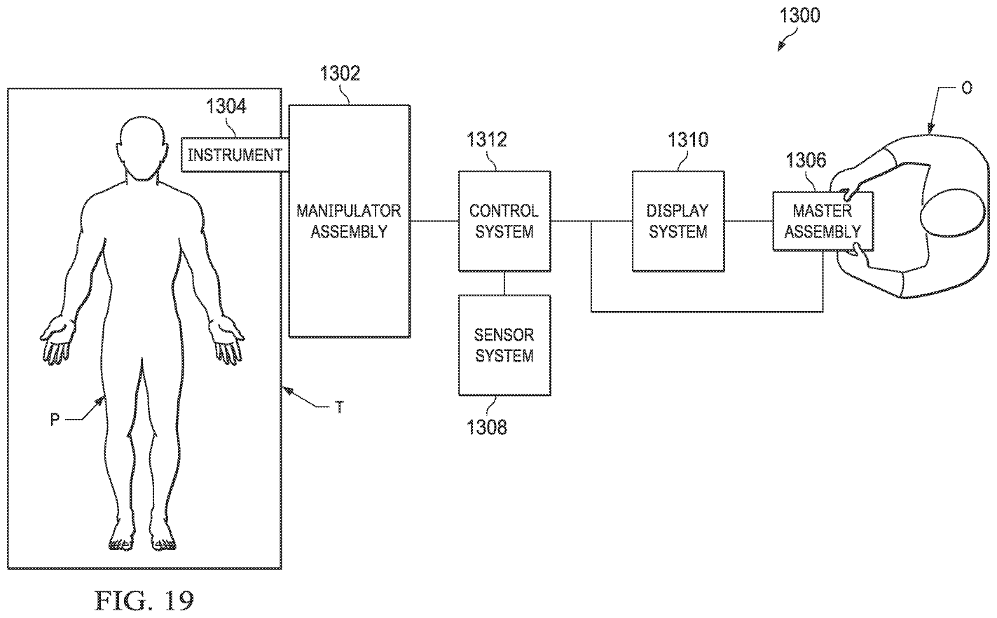

As shown in FIG. 19, a medical system 1300 generally includes a manipulator assembly 1302 (e.g., the manipulator assembly 110) for operating a medical instrument 1304 (e.g., the medical instrument 120) in performing various procedures on a patient P positioned on a table T. The manipulator assembly 1302 may be robotic-assisted, non-robotic-assisted, or a hybrid robotic-assisted and non-robotic-assisted assembly with select degrees of freedom of motion that may be motorized and/or robotic-assisted and select degrees of freedom of motion that may be non-motorized and/or non-robotic-assisted. The medical system 1300 may further include a master assembly 1306, which generally includes one or more control devices for controlling manipulator assembly 1302. Manipulator assembly 1302 supports medical instrument 1304 and may optionally include a plurality of actuators or motors that drive inputs on medical instrument 1304 in response to commands from a control system 1312. The actuators may optionally include drive systems that when coupled to medical instrument 1304 may advance medical instrument 1304 into a naturally or surgically created anatomic orifice.

Medical system 1300 also includes a display system 1310 for displaying an image or representation of the surgical site and medical instrument 1304 generated by sub-systems of sensor system 1308. Display system 1310 and master assembly 1306 may be oriented so operator O can control medical instrument 1304 and master assembly 1306 with the perception of telepresence. Additional information regarding the medical system 1300 and the medical instrument 1304 may be found in International Application Publication No. WO 2018/195216, filed on Apr. 18, 2018, entitled “Graphical User Interface for Monitoring an Image-Guided Procedure,” which is incorporated by reference herein in its entirety.

In some examples, medical instrument 1304 may include components of an imaging system (discussed in more detail below), which may include an imaging scope assembly or imaging instrument that records a concurrent or real-time image of a surgical site and provides the image to the operator or operator O through one or more displays of medical system 1300, such as one or more displays of display system 1310. The concurrent image may be, for example, a two or three-dimensional image captured by an imaging instrument positioned within the surgical site. In some examples, the imaging system includes endoscopic imaging instrument components that may be integrally or removably coupled to medical instrument 1304. However, in some examples, a separate endoscope, attached to a separate manipulator assembly may be used with medical instrument 1304 to image the surgical site. In some examples, as described in detail below, the imaging instrument alone or in combination with other components of the medical instrument 1304 may include one or more mechanisms for cleaning one or more lenses of the imaging instrument when the one or more lenses become partially and/or fully obscured by fluids and/or other materials encountered by the distal end of the imaging instrument. In some examples, the one or more cleaning mechanisms may optionally include an air and/or other gas delivery system that is usable to emit a puff of air and/or other gasses to blow the one or more lenses clean. Examples of the one or more cleaning mechanisms are discussed in more detail in International Application Publication No. WO/2016/025465, filed on Aug. 11, 2016, entitled “Systems and Methods for Cleaning an Endoscopic Instrument”; U.S. patent application Ser. No. 15/508,923, filed on Mar. 5, 2017, entitled “Devices, Systems, and Methods Using Mating Catheter Tips and Tools”; and U.S. patent application Ser. No. 15/503,589, filed Feb. 13, 2017, entitled “Systems and Methods for Cleaning an Endoscopic Instrument,” each of which is incorporated by reference herein in its entirety. The imaging system may be implemented as hardware, firmware, software or a combination thereof which interact with or are otherwise executed by one or more computer processors, which may include the processors of the control system 1312.

Control system 1312 includes at least one memory and at least one computer processor (not shown) for effecting control between medical instrument 1304, master assembly 1306, sensor system 1308, and display system 1310. Control system 1312 also includes programmed instructions (e.g., a non-transitory machine-readable medium storing the instructions) to implement some or all of the methods described in accordance with aspects disclosed herein, including instructions for providing information to display system 1310.

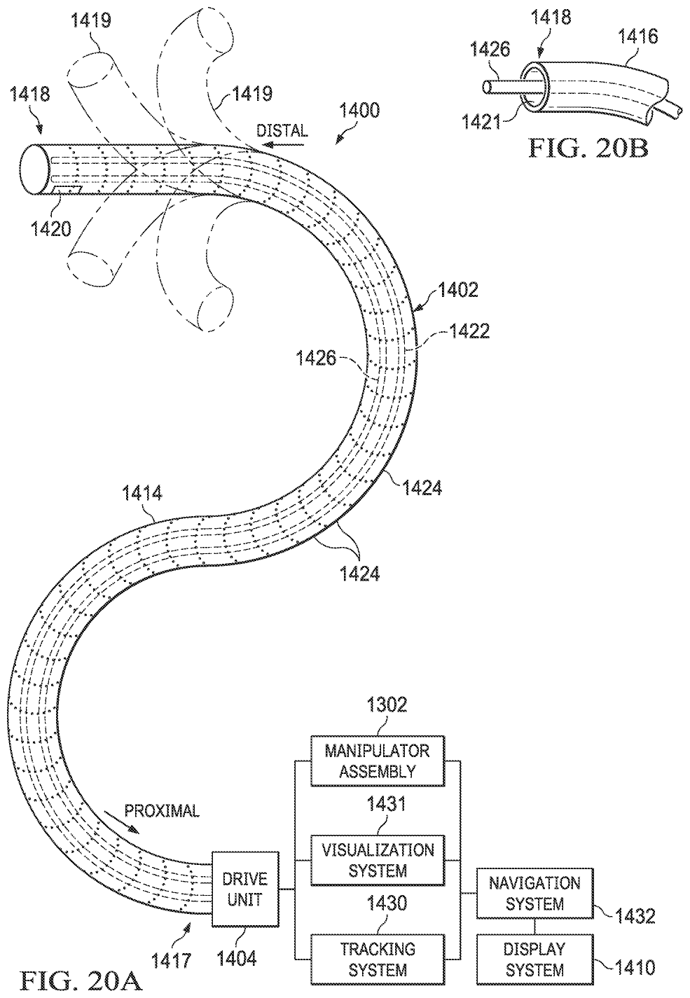

FIG. 20A is a simplified diagram of a medical instrument system 1400 according to some examples. Medical instrument system 1400 includes elongate device 1402, such as a flexible catheter (e.g., the elongate device 122), coupled to a drive unit 1404. Elongate device 1402 includes a flexible body 1416 having proximal end 1417 and distal end or tip portion 1418. Medical instrument system 1400 further includes a tracking system 1430 for determining the position, orientation, speed, velocity, pose, and/or shape of distal end 1418 and/or of one or more segments 1424 along flexible body 1416 using one or more sensors and/or imaging devices as described in further detail below.

Tracking system 1430 may optionally track distal end 1418 and/or one or more of the segments 1424 using a shape sensor 1422. Shape sensor 1422 may optionally include an optical fiber aligned with flexible body 1416 (e.g., provided within an interior channel (not shown) or mounted externally). The optical fiber of shape sensor 1422 forms a fiber optic bend sensor for determining the shape of flexible body 1416. In one alternative, optical fibers including Fiber Bragg Gratings (FBGs) are used to provide strain measurements in structures in one or more dimensions. Various systems and methods for monitoring the shape and relative position of an optical fiber in three dimensions are described in U.S. patent application Ser. No. 11/180,389, filed on Jul. 13, 2005, entitled “Fiber Optic Position and Shape Sensing Device and Method Relating Thereto”; U.S. patent application Ser. No. 12/047,056, filed on Jul. 16, 2004, entitled “Fiber-Optic Shape and Relative Position Sensing”; and U.S. Pat. No. 6,389,187, filed on Jun. 17, 1998, entitled “Optical Fibre Bend Sensor”, each of which is incorporated by reference herein in its entirety. Sensors in some examples may employ other suitable strain sensing techniques, such as Rayleigh scattering, Raman scattering, Brillouin scattering, and Fluorescence scattering. In some examples, the shape of the elongate device may be determined using other techniques. For example, a history of the distal end pose of flexible body 1416 can be used to reconstruct the shape of flexible body 1416 over the interval of time. In some examples, tracking system 1430 may optionally and/or additionally track distal end 1418 using a position sensor system 1420. Position sensor system 1420 may be a component of an EM sensor system with position sensor system 1420 including one or more conductive coils that may be subjected to an externally generated electromagnetic field. Each coil of the EM sensor system then produces an induced electrical signal having characteristics that depend on the position and orientation of the coil relative to the externally generated electromagnetic field. In some examples, position sensor system 1420 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732, filed on Aug. 11, 1999, entitled “Six-Degree of Freedom Tracking System Having a Passive Transponder on the Object Being Tracked”, which is incorporated by reference herein in its entirety.

Flexible body 1416 includes a channel 1421 sized and shaped to receive a medical instrument 1426. FIG. 20B is a simplified diagram of flexible body 1416 with medical instrument 1426 extended according to some examples. In some examples, medical instrument 1426 may be used for procedures such as surgery, biopsy, ablation, illumination, irrigation, or suction. Medical instrument 1426 can be deployed through channel 1421 of flexible body 1416 and used at a target location within the anatomy. Medical instrument 1426 may include, for example, image capture probes, biopsy instruments, laser ablation fibers, and/or other surgical, diagnostic, or therapeutic tools. Medical instrument 1426 may be used with an imaging instrument (e.g., an image capture probe) also within flexible body 1416. The imaging instrument may include a cable coupled to the camera for transmitting the captured image data. In some examples, the imaging instrument may be a fiber-optic bundle, such as a fiberscope, that couples to image processing system 1431. The imaging instrument may be single or multi-spectral, for example capturing image data in one or more of the visible, infrared, and/or ultraviolet spectrums. Medical instrument 1426 may be advanced from the opening of channel 1421 to perform the procedure and then retracted back into the channel when the procedure is complete. Medical instrument 1426 may be removed from proximal end 1417 of flexible body 1416 or from another optional instrument port (not shown) along flexible body 1416.

Flexible body 1416 may also house cables, linkages, or other steering controls (not shown) that extend between drive unit 1404 and distal end 1418 to controllably bend distal end 1418 as shown, for example, by broken dashed line depictions 1419 of distal end 1418. In some examples, at least four cables are used to provide independent “up-down” steering to control a pitch of distal end 1418 and “left-right” steering to control a yaw of distal end 1418. Steerable elongate devices are described in detail in U.S. patent application Ser. No. 13/274,208, filed on Oct. 14, 2011, entitled “Catheter with Removable Vision Probe”, which is incorporated by reference herein in its entirety.

The information from tracking system 1430 may be sent to a navigation system 1432 where it is combined with information from image processing system 1431 and/or the preoperatively obtained models to provide the operator with real-time position information. In some examples, the real-time position information may be displayed on display system 1310 of FIG. 19 for use in the control of medical instrument system 1400. In some examples, control system 1312 of FIG. 19 may utilize the position information as feedback for positioning medical instrument system 1400. Various systems for using fiber optic sensors to register and display a surgical instrument with surgical images are provided in U.S. patent application Ser. No. 13/107,562, filed on May 13, 2011, entitled “Medical System Providing Dynamic Registration of a Model of an Anatomic Structure for Image-Guided Surgery,” which is incorporated by reference herein in its entirety.

In some examples, medical instrument system 1400 may be robotic-assisted within medical system 1300 of FIG. 19. In some examples, manipulator assembly 1302 of FIG. 19 may be replaced by direct operator control. In some examples, the direct operator control may include various handles and operator interfaces for hand-held operation of the instrument.

The singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

In the description, specific details have been set forth describing some embodiments. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately. For simplicity, in some instances the same reference numbers are used throughout the drawings to refer to the same or like parts.

The systems and methods described herein may be suited for navigation and treatment of anatomic tissues, via natural or surgically created connected passageways, in any of a variety of anatomic systems, including the lung, colon, the intestines, the kidneys and kidney calices, the brain, the heart, the circulatory system including vasculature, and/or the like. Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy), and performing procedures on human or animal cadavers. Further, these techniques can also be used for surgical and nonsurgical medical treatment or diagnosis procedures.

Further, although some of the examples presented in this disclosure discuss robotic-assisted systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium (e.g., a non-transitory storage medium) or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain example examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. An imaging coupler comprising:
an elongate device connector configured to couple to an elongate device;
an instrument connector configured to couple to an imaging instrument, the imaging instrument configured to be slidably received within a lumen of the elongate device;
a body portion extending between the elongate device connector and the instrument connector; and
a tubular member coupled to the instrument connector and extending within the body portion,
wherein the instrument connector is movable in parallel with a longitudinal axis of the tubular member while the elongate device is coupled to the elongate device connector.

2. The imaging coupler of claim 1, wherein the tubular member includes an inner surface defining a lumen configured to slidably receive the imaging instrument.

3. The imaging coupler of claim 1, wherein when the instrument connector moves in a proximal direction, a distal end of the imaging instrument moves in the proximal direction and is retracted within the lumen of the elongate device.

4. The imaging coupler of claim 1, wherein when the instrument connector moves, the elongate device remains stationary.

5. The imaging coupler of claim 1, wherein the tubular member includes a ring at a distal end of the tubular member for preventing removal of the tubular member from the body portion.

6. (canceled)

7. The imaging coupler of claim 1, wherein the imaging coupler further comprises a biasing member coupled to the tubular member.

8. The imaging coupler of claim 7, wherein the biasing member biases the tubular member in a distal direction.

9-34. (canceled)

35. The imaging coupler of claim 1, wherein a distal end of the instrument connector includes a locking member, wherein a proximal end of the body portion includes a recess in a wall of the body portion, and wherein the locking member is configured to be received by the recess.

36. The imaging coupler of claim 35, wherein the instrument connector is configured to be rotated in a first rotational direction to couple the instrument connector and the body portion, and wherein the instrument connector is configured to be rotated in a second rotational direction to decouple the instrument connector and the body portion.

37. The imaging coupler of claim 36, wherein the first rotational direction is a clockwise direction, and wherein the second rotational direction is a counterclockwise direction.

38. The imaging coupler of claim 35, wherein the locking member includes an elongate portion and a hook portion extending from the elongate portion.

39. The imaging coupler of claim 38, wherein the recess includes an entry recess and a locking recess, and wherein the hook portion is configured to be received within the locking recess.

40. The imaging coupler of claim 39, wherein the hook portion is configured to be received within the locking recess when the instrument connector is rotated in a clockwise direction.

41. The imaging coupler of claim 39, wherein when the hook portion is received within the locking recess, the imaging coupler is in a locked configuration.

42. (canceled)

43. A system comprising:
an imaging instrument configured to be slidably received within a lumen of an elongate device; and
an imaging coupler including proximal and distal portions, the imaging coupler comprising:
an elongate device connector configured to couple to the elongate device;
an instrument connector configured to couple to the imaging instrument;
a body portion extending between the elongate device connector and the instrument connector; and
a tubular member coupled to the instrument connector and extending within the body portion,
wherein the instrument connector is movable in parallel with a longitudinal axis of the tubular member while the elongate device is coupled to the elongate device connector.

44. The system of claim 43, wherein when the instrument connector moves in a proximal direction, a distal end of the imaging instrument moves in the proximal direction and is retracted within the lumen of the elongate device.

45. The system of claim 43, wherein when the instrument connector moves, the elongate device remains stationary.

46-61. (canceled)

62. The system of claim 43, wherein a distal end of the instrument connector includes a locking member, wherein a proximal end of the body portion includes a recess in a wall of the body portion, and wherein the locking member is configured to be received by the recess.

63. The imaging coupler of claim 62, wherein the instrument connector is configured to be rotated in a first rotational direction to couple the instrument connector and the body portion, and wherein the instrument connector is configured to be rotated in a second rotational direction to decouple the instrument connector and the body portion.

64. The imaging coupler of claim 63, wherein the locking member includes an elongate portion and a hook portion extending from the elongate portion, wherein the recess includes an entry recess and a locking recess, and wherein the hook portion is configured to be received within the locking recess.

65-139. (canceled)
